(19) United States
(12) Patent Application Publication
(10) Pub. No.: US 2021/0024583 A1
(43) Pub. Date: Jan. 28, 2021

(54) COMPOSITIONS AND METHODS FOR PEPTIDE MEDIATED OLIGONUCLEOTIDE DELIVERY TO THE CENTRAL NERVOUS SYSTEM

(71) Applicant: The Regents of the University of California, Oakland, CA (US)

(72) Inventors: Brian SPENCER, LA Jolla, CA (US); Robert RISSMAN, San Diego, CA (US)

(21) Appl. No.: 15/931,182

(22) Filed: May 13, 2020

Related U.S. Application Data
(60) Provisional application No. 62/847,069, filed on May 13, 2019.

(51) Int. Cl. C07K 14/00; A61K 9/00; A61K 48/00; A61P 25/28

(52) U.S. Cl. CPC C07K 14/003; A61P 25/28; A61K 48/0041; A61K 9/0019

(57) ABSTRACT

In alternative embodiments, provided are compositions, including products of manufacture and kits, and methods, for transporting nucleic acids such as oligonucleotides to and across the blood brain barrier (BBB) and into the central nervous system (CNS). In alternative embodiments, provided herein are ApoB11-comprising chimeric or synthetic peptides useful for the delivery of nucleic acids such as oligonucleotides to the CNS for the treatment, prevention or amelioration of a CNS disease, infection or condition, or for diagnostic purposes.

### RELATED APPLICATIONS

This U.S. utility patent application claims the benefit of priority to U.S. Provisional Patent Application Serial No. (USSN) 62/847,069 filed May 13, 2019. The aforementioned application is expressly incorporated herein by reference in its entirety and for all purposes.

### STATEMENT AS TO FEDERALLY SPONSORED RESEARCH

This invention was made with government support under National Institutes of Health (NIH), DHHS, grant nos. AG05131 and AG18440. The government has certain rights in the invention.

### TECHNICAL FIELD

This invention generally relates to neuroscience, drug delivery and neurology. In alternative embodiments, provided are compositions, including products of manufacture and kits, and methods, for transporting nucleic acids such as oligonucleotides to and across the blood brain barrier (BBB) and into the central nervous system (CNS). In alternative embodiments, provided herein are ApoB11-comprising chimeric or synthetic peptides useful for the delivery of nucleic acids such as oligonucleotides to the CNS for the treatment, prevention or amelioration of a CNS disease, infection or condition, or for diagnostic purposes.

### BACKGROUND

Many diseases of the central nervous system arise from the accumulation of protein such as α-synuclein in Parkinson's Disease or Aβ in Alzheimer's disease. The ability to regulate the expression at the gene transcription level would be beneficial for reducing the accumulation of these proteins or regulating expression levels of other genes in the CNS. Short interfering RNA molecules can bind specifically to target RNAs and deliver them for degradation. This approach has shown promise therapeutically in vitro and in vivo in mouse models of PD and AD and other neurological disorders; however, delivery of the siRNA to the CNS in vivo has been achieved primarily through intra-cranial stereotaxic injection. Repeat injections by stereo-taxic injections may not be amenable to clinical translation; therefore, a new approach for delivery of siRNAs to the brain is needed.

Recently, elAgnaf et al identified a small peptide from the envelope protein of the rabies virus that could deliver a siRNA via intra-venous delivery to the brain utilizing a receptor on the blood-brain barrier. However, this receptor is saturable and will only allow the delivery of a limited number of molecules.

The blood-brain barrier (BBB) controls the passage of substances from the blood into the central nervous system. Small molecules (400-500 Daltons) can pass freely; however, larger molecules are actively transported across the BBB via three main mechanisms: (a) carrier mediated transporters (CMT) allow the transport of nutrients such as sugars and amino acids from blood to the brain, (b) receptor mediated transport (RMT) allows the transport of larger proteins and carrier proteins such as transferrin (iron), apolipoprotein (lipids) and insulin from the blood to the brain, and (c) efflux transporters export drugs from the CNS to the blood e.g. p-glycoprotein and breast cancer resistance protein²⁶⁻²⁸. Thus, a major challenge for the delivery of oligonucleotides for the treatment of CNS diseases such as neurodegenerative diseases is transport of therapeutics into the CNS.

The low-density lipoprotein receptor family is a group of cell surface receptors that bind lipoprotein complexes for internalization to the lysosomes. The family comprises approximately ten different receptors with the most common examples being low-density lipoprotein receptor (LDLR), low-density lipoprotein related receptor (LRP), very-low density lipoprotein receptor (VLDL), megalin and apolipoprotein E receptor. The receptors are expressed in a tissue specific manner and primarily bind apolipoprotein complexes. The apolipoprotein, of which the two most prominent members are apolipoprotein B (ApoB) and apolipoprotein E (ApoE), function to bind lipids in the blood stream and target them for lysosomal degradation. Binding of the apolipoproteins to the receptor results in endocytosis and transport to the lysosome where the low pH compartment facilitates the release of the protein complex. The LDL receptor is then recycled to the cell surface. At the blood brain barrier, the LDL receptor binds lipoproteins resulting in endocytosis. Following endocytosis by the endothelial cells, a portion of the LDL receptor is shuttled to the apical side of the BBB where presumably, the apolipoprotein is released to be taken up by neurons and/or astrocytes.

### SUMMARY

In alternative embodiments, provided chimeric or synthetic peptides, comprising:

(a) a first peptide comprising an amino acid sequence as set forth in SEQ ID NO:2, or a mimetic or peptidomimetic thereof;

(b) a second peptide comprising at least 2, 3, 4, 5, 6, 7, 8, 9 or 10 or more or a plurality of amino acids or mimetics or peptidomimetics thereof capable of functioning as a flexible linker,

and optionally the second peptide comprises at least 2, 3, 4, 5, 6, 7, 8, 9 or 10 or more or a plurality of glycines, or mixture of glycines, serines, valines, alanines, isoleucines, leucines or mimetics or peptidomimetics thereof,

and optionally the second peptide comprises or consists of five glycines or mixture of glycines and serines, or mixture of glycines, serines, valines, alanines, isoleucines, leucines or mimetics or peptidomimetics thereof,

and optionally the second peptide comprises or consists of GSGGG (SEQ ID NO:7), GGSGG (SEQ ID NO:8), GGGGS (SEQ ID NO:9), GGSGGG (SEQ ID NO:10), GSGGGG (SEQ ID NO:11), GGSGGGS (SEQ ID NO:12), GGSGSGS (SEQ ID NO:13), GGSGSGSGG (SEQ ID NO:14), GGSGGGSGG (SEQ ID NO:15), GGSGGGSGGGS (SEQ ID NO:16) or GGSGGGSGSGS (SEQ ID NO:17) or mimetics or peptidomimetics thereof; and

(c) a positively charged third peptide comprising at least 2, 3, 4, 5, 6, 7, 8, 9, 10, 11, 12, 13 or 14 or more positively charged amino acids, or mimetics or peptidomimetics thereof, wherein optionally the positively charged amino acids comprise an arginine or a mimetic or peptidomimetic thereof, and optionally the third peptide comprises nine arginines or mimetics or peptidomimetics thereof,

wherein optionally the chimeric or synthetic peptide comprises or consists of: RLTRKRGLKLAGGGGGRRRRRRRRR (SEQ ID NO:3) or a mimetic or peptidomimetic thereof.

In alternative embodiments of the chimeric or synthetic peptides as provided herein:
the chimeric or synthetic peptide further comprises a nucleic acid, or a negatively charged payload, wherein optionally the nucleic acid or the negatively charged payload is electrostatically bound, or bound by a charge, to the positively charged third peptide, or the nucleic acid or negatively charged payload is bound by a chemical bond to the chimeric or synthetic peptide
the first peptide is linked to the second peptide by a peptide bond; or, the second peptide and the positively charged third peptide are linked together by a peptide bond; or, the first peptide, the second peptide and the positively charged third peptide are all linked together by a peptide bond;
the nucleic acid is or comprises an natural or a synthetic oligonucleotide, and optionally the natural or synthetic oligonucleotide comprises an RNA or a DNA, and optionally the natural or synthetic oligonucleotide comprises a small inhibitory RNA (siRNA), a cDNA, an mRNA, an shRNA, an miRNA or a gRNA, and optionally the deoxyribonucleic acid (DNA) comprises an antisense oligonucleotide, and optionally the antisense oligonucleotide comprises a modified or synthetic anti-sense oligonucleotide (ASO), and optionally the modified or synthetic anti-sense oligonucleotide comprises: a 2′-O-(2-MethoOxyEthyl)-oligoribonucloeotide (2′-MOE); a 2′-O-Methyl-RNA (2′-OMe) oligoribonucloeotide; a phosphorothioate DNA or an oligonucleotide phosphorothioate (OPS); a phosphorodiamidate morpholino oligomer (PMO); a tricyclo-DNA or tcDNA; a locked nucleic acid (LNA) or inaccessible RNA (or RNA nucleotide having a ribose moiety modified with an extra bridge connecting a 2′ oxygen and a 4′ carbon); a morpholino, 5′ methylcytosine base; and/or other modified nucleic acids;
the chimeric or synthetic peptide comprises at least one synthetic amino acid, or at least two amino acid are joined by at least one non-peptide (non-amide) bond, and optionally the at least one non-peptide comprises an isopeptide bond; and/or
the chimeric or synthetic peptide is formulated as a sterile solution, e.g., a sterile saline or buffered saline solution, or as an injectable formulation.

In alternative embodiments, provided are products of manufacture comprising a chimeric or synthetic peptide as provided herein, wherein the product of manufacture comprises an implant, an ampoule or a syringe.

In alternative embodiments, provided are methods for treating, preventing, ameliorating and/or diagnosing a disease, infection or condition of the central nervous system (CNS) in an individual in need thereof, or causing a neuro-modulatory treatment or effect on the individual in need thereof, comprising: administering to the individual in need thereof a chimeric or synthetic peptide as provided herein, wherein the chimeric or synthetic peptide or synthetic further comprises or is bound to a nucleic acid capable of treating, preventing, ameliorating and/or diagnosing the disease or condition of the CNS in the individual in need thereof. In alternative embodiments of the methods, the disease, infection or condition of the CNS is or causes a neurodegenerative disease or a cancer; or, the neurodegenerative disease or CNS condition is: Alzheimer's disease, Lewy Body (DLB), multiple system atrophy (MSA), Gaucher's disease, amyotrophic lateral sclerosis (ALS), neuronal ceroid lipofuscinosis (NCL) (Batten disease), Parkinson's disease (PD), spinocerebellar ataxia (SCA) frontotemporal lobar degeneration (Pick's disease), a prion disease (Creutzfeldt-Jakob Disease) or Huntington's disease.

In alternative embodiments, provided are methods for gene targeted editing, or for genomic engineering, epigenomic engineering and/or genome editing, of the CNS, comprising: administering to the individual in need thereof a chimeric or synthetic peptide or synthetic as provided herein, wherein the chimeric or synthetic peptide or synthetic further comprises or is bound to a nucleic acid capable of gene targeted editing, for example, for genomic engineering, epigenomic engineering, genome targeting, and/or genome editing, and optionally the gene targeted editing comprises a CRISPR (clustered regularly interspaced short palindromic repeats) encoding nucleic acid or equivalent gene editing nucleic acid sequence, such as a CRISPR-Cas9 nuclease encoding nucleic acid, or a gRNA molecule, for example, as described in U.S. Pat. No. 10,633,642 (describing engineered CRISPR-Cas9 nucleases); U.S. Pat. No. 10,479,982 (describing engineered CRISPR-Cas9 nucleases with altered PAM specificity); U.S. Pat. No. 10,385,336 (describing programmable RNA shredding by the type III-A CRISPR-Cas system); U.S. Pat. No. 9,834,791. In alternative embodiment, the nucleic acid payload comprises a guide RNA, a crRNA and/or a tracrRNA.

In alternative embodiments, of the methods, the chimeric or synthetic peptide or synthetic is formulated as a sterile injectable formulation, and optionally the chimeric or synthetic peptide is administered to the individual in need thereof by intraperitoneal (IP), intravenous (IV), intramuscular (IM) or intrathecal injection, or by inhalation.

In alternative embodiments, provided are uses of a chimeric or synthetic peptide as provided herein, or a kit as provided herein, for gene targeted editing of the CNS, or for treating, preventing, ameliorating and/or diagnosing a disease, infection or condition of the central nervous system (CNS) in an individual in need thereof, or causing a neuro-modulatory treatment or effect on an individual in need thereof.

In alternative embodiments, provided are chimeric or synthetic peptides as provided herein, or a kit as provided herein, for use in gene targeted editing of the CNS, or for treating, preventing, ameliorating and/or diagnosing a disease, infection or condition of the central nervous system (CNS) in an individual in need thereof, or causing a neuro-modulatory treatment or effect on an individual in need thereof.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The drawings set forth herein are illustrative of exemplary embodiments provided herein and are not meant to limit the scope of the invention as encompassed by the claims.

FIG. 1A-F illustrate that an exemplary non-viral peptide as provided herein that can deliver a short RNA to neuronal cells:

FIG. 1A illustrates an exemplary 11 amino acid (underlined) fragment, called “ApoB¹¹ peptide”, coupled to a 5 Glycine linker and a 9 Arginine positively charged tail; where the exemplary ApoB³⁸ peptide VDSSVIDALQYKLEGTTRLTRKRGLKLAGGGGGRRRRRRRRR is SEQ ID NO:1; where the apoB¹¹ sequence/motif is RLTRKRGLKLA is SEQ ID NO:2; and, the exemplary peptide RLTRKRGLKLAGGGGGRRRRRRRRR is SEQ ID NO:3;

FIG. 1B illustrates an agarose gel of siRNA that was incubated with different molar ratios of the ApoB¹¹ peptide for 30 minutes; unconjugated RNA was observed by ethidium bromide staining;

FIG. 1C and FIG. 1E illustrate images of fluorescein isothiocyanate (FITC) labeled siRNA that were added to N2A mouse neuronal cells at a ratio of 1:10 siRNA:ApoB¹¹ or 1:40 siRNA:C2-9r and analyzed at different time points for uptake of the FITC labeled siRNA;

FIG. 1D and FIG. 1F graphically illustrate FITC signal, or pixel intensity, from coverslips analyzed for siRNA (FITC signal) pixel intensity; scale bar represents 15 μm,

as discussed in detail in Example 1, below.

FIG. 2A-F illustrate that peptide delivery of siRNA targeting α-synuclein reduced the accumulation of α-synuclein in neuronal cells in vitro:

FIG. 2A, FIG. 2C illustrate images of N2A neuronal cells treated with 100 pmol siαSyn conjugated to the (FIG. 2A) C2-9r peptide or to the (FIG. 2C) ApoB¹¹ peptide and immunostained for α-synuclein and the neuronal marker MAP2;

FIG. 2B, FIG. 2D graphically illustrate pixel intensity from coverslips analyzed for α-synuclein;

FIG. 2E illustrates a representative immunoblot from replicate from N2A cultures treated with siαSyn conjugated to the C2-9r peptide or the ApoB¹¹ peptide;

FIG. 2F graphically illustrates a densitometry analysis of the α-synuclein bands as a ratio to β-actin, scale bar represents 10 μm, as discussed in detail in Example 1, below.

FIG. 3A-F illustrate that peptide-siRNA is distributed to the brain following i.p. delivery, where non-tg and α-syn tg mice received a single i.p. injection of fluorescein isothiocyanate (FITC) labeled α-syn siRNA conjugated to either C2-9r, ApoB¹¹ or naked and 4 hours after injection, mice were sacrificed, and whole brain was removed and fixed:

FIG. 3A, FIG. 3B illustrate sections were stained for the neuronal marker NeuN and the nuclear stain DAPI and then visualized for co-localization with the FITC labeled siRNA;

FIG. 3D, FIG. 3E illustrate sections were stained for the astrocytic marker GFAP and the nuclear stain DAPI and then visualized for co-localization with the FITC labeled siRNA;

FIG. 3C, FIG. 3F graphically illustrate analysis of % co-localization between (FIG. 3C) NeuN (red) and siRNA (green) or (FIG. 3F) GFAP (red) and siRNA (green),

as discussed in detail in Example 1, below.

FIG. 4A-C illustrate images showing that peptide-siRNA co-localizes with and reduces the accumulation of α-synuclein in the brain following i.p. delivery:

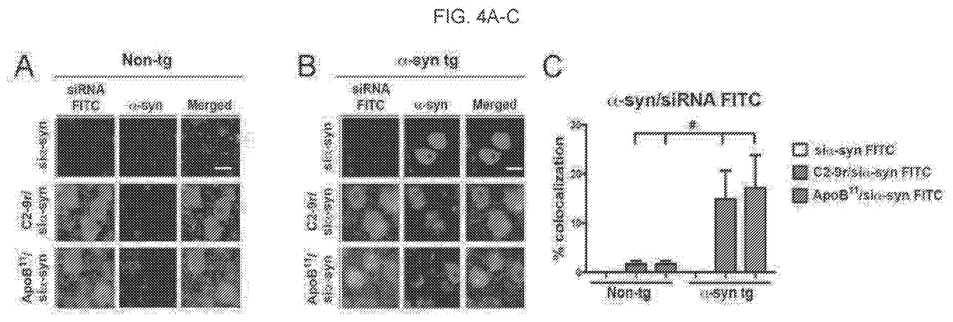

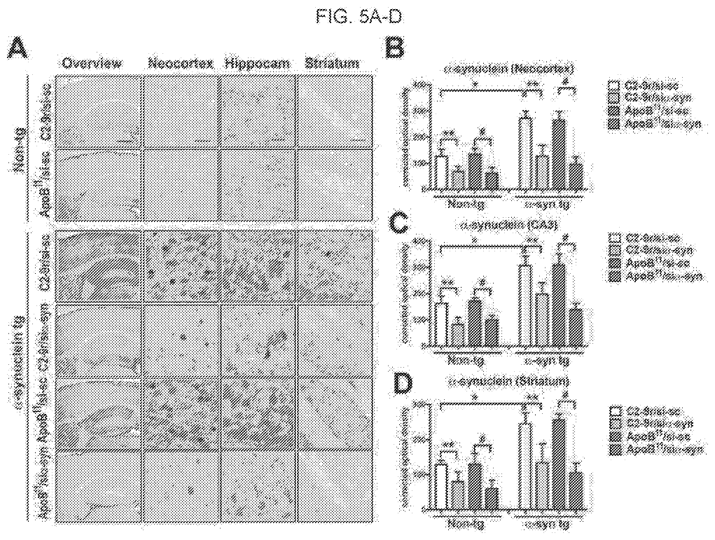

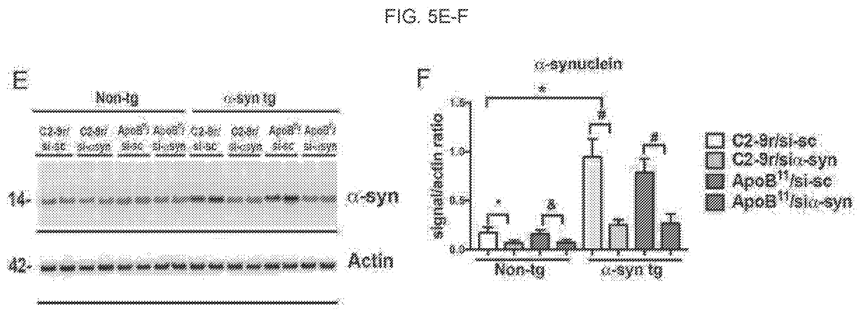

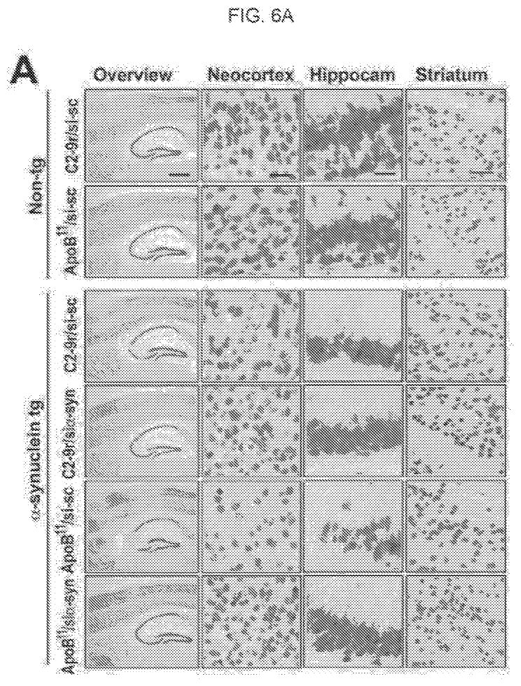

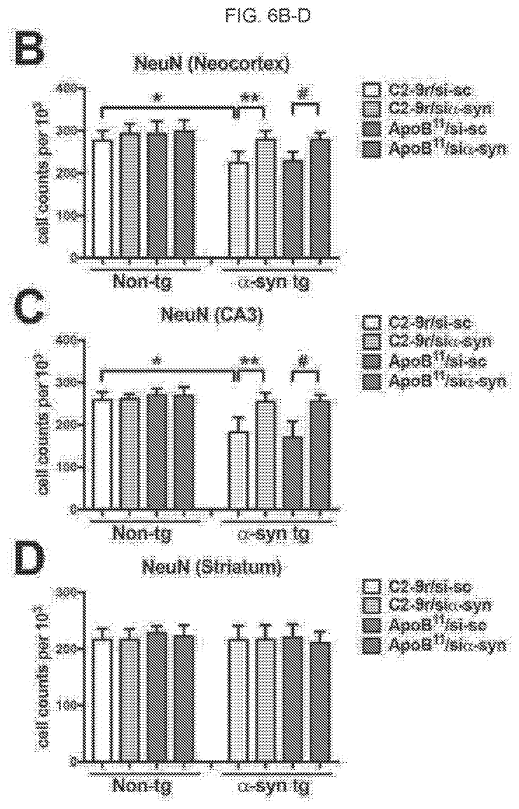

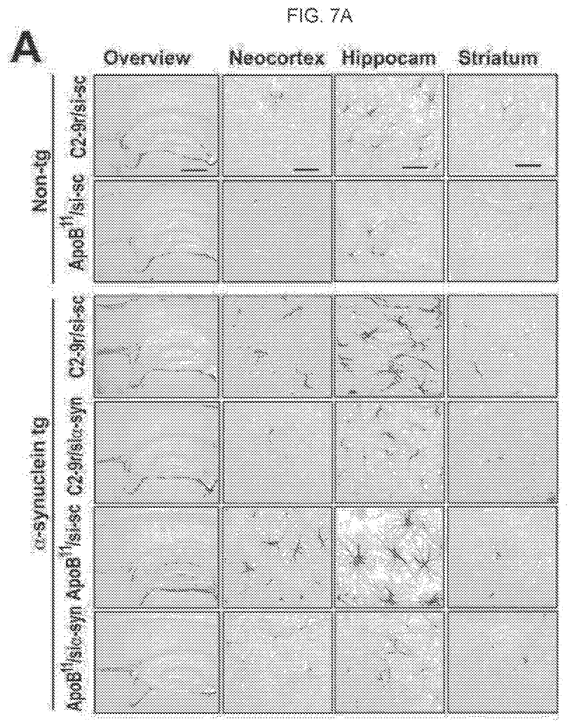

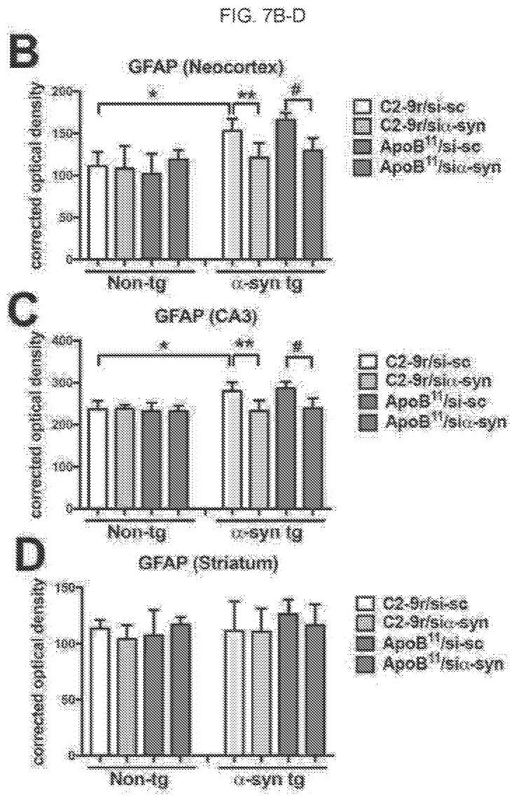

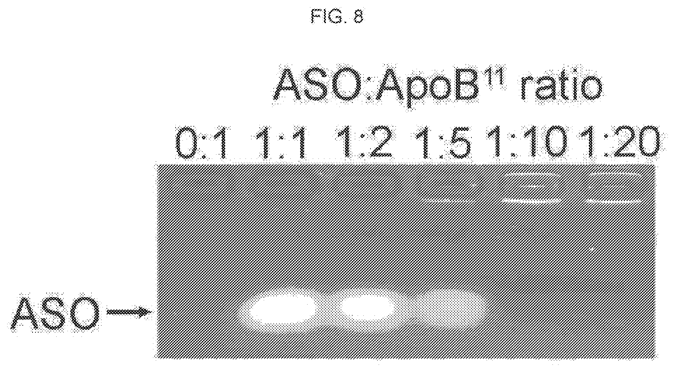

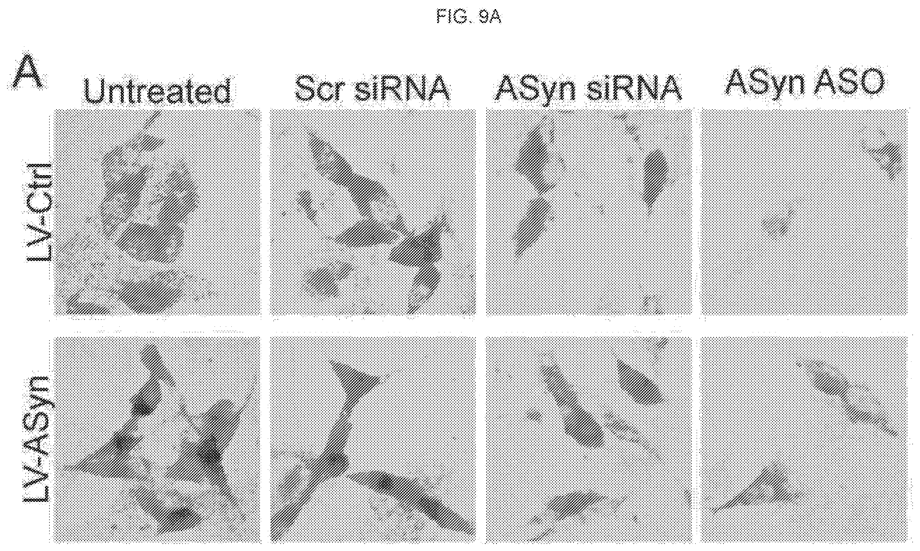

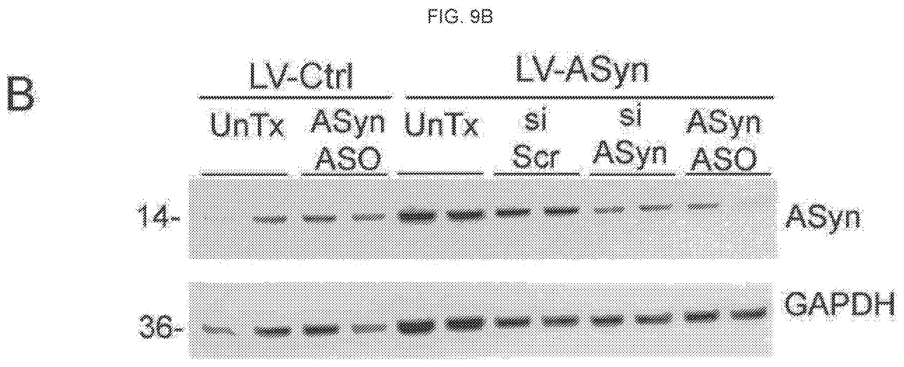

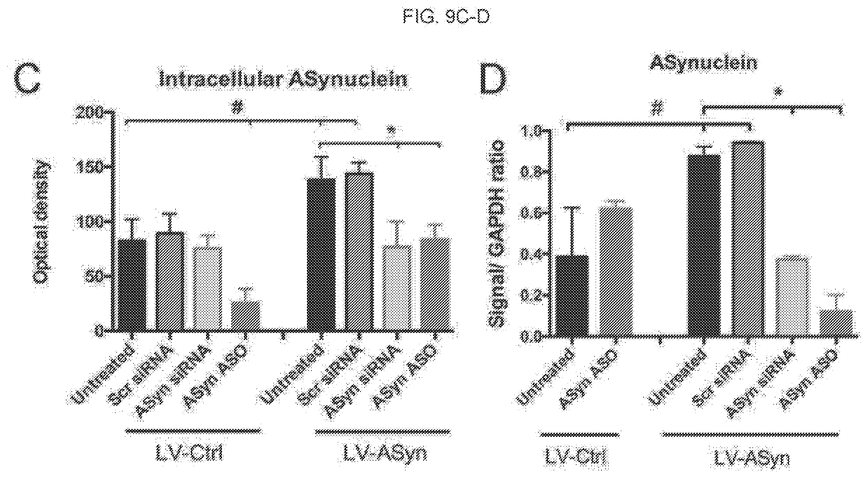

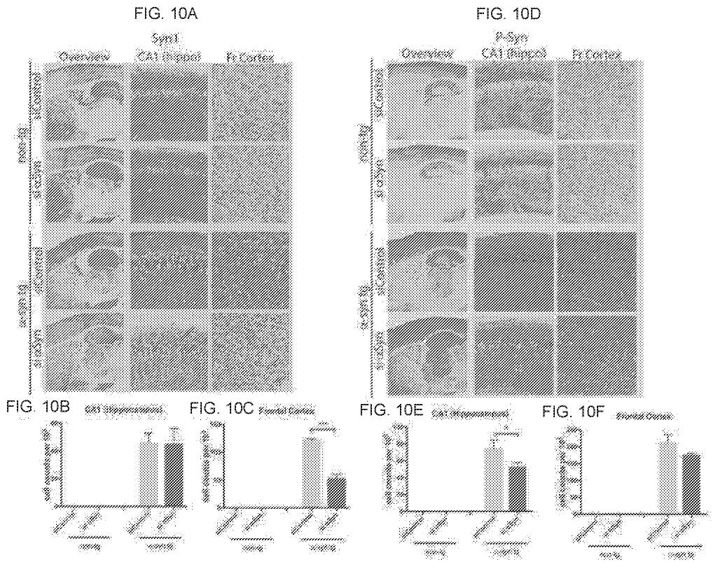

Non-tg (FIG. 4A) and α-syn tg (FIG. 4B) mice received a single i.p. injection of fluorescein isothiocyanate (FITC) labeled α-syn siRNA conjugated to either C2-9r, ApoB¹¹ or naked, 4 hours after injection, mice were sacrificed, whole brain removed and fixed, and sections were stained for α-synuclein and the nuclear marker, DAPI and then visualized for co-localization with the FITC labeled siRNA, FIG. 4C graphically summarizes the fluorescent reading of FIG. 4A and FIG. 4B, showing the percent (%) co-localization of FITC labeled siαSyn conjugated to either ApoB 11 or C2-9r and αSyn

as discussed in detail in Example 1, below.

FIG. 5A-F illustrate that systemic delivery of the siαSyn conjugated to C2-9r or ApoB¹¹ peptides reduces the accumulation of α-synuclein in the brain of transgenic mice, where 3-month-old α-syn tg and non-tg mice received a repeat i.p. injections of α-syn siRNA or scrambled siRNA conjugated to either C2-9r, ApoB¹¹ and were sacrificed 2 weeks later for analysis:

FIG. 5A illustrates images of immunocytochemical analysis with antibodies against the α-synuclein in brain sections from the neocortex, hippocampus and striatum of non-tg and α-syn tg mice;

FIG. 5B-D graphically illustrate image analysis of levels of expressed as corrected optical density for α-synuclein immunoreactivity in the (FIG. 5B) neocortex, (FIG. 5C) hippocampus (CA3), and (FIG. 5D) striatum;

FIG. 5E illustrates an image of a representative, immunoblot analysis of brain homogenates from non-tg and αsyn tg mice treated with α-syn siRNA or scrambled siRNA conjugated to either C2-9r, ApoB¹¹;

FIG. 5F graphically illustrates an image analysis of pixels of α-synuclein immunoreactivity analyzed as ratio to β-actin signal,

as discussed in detail in Example 1, below.

FIG. 6A-D illustrate that systemic delivery of the si-αSyn conjugated to C2-9r or ApoB¹¹ peptides reduces neurodegeneration in the brain of α-syn tg mice, wherein 3-month-old α-syn tg and non-tg mice received repeat i.p. injections of α-syn siRNA or scrambled siRNA conjugated to either C2-9r, ApoB¹¹ and were sacrificed 2 weeks later for analysis:

FIG. 6A illustrates images of an immunocytochemical analysis with antibodies against the neuronal marker, NeuN in brain sections from the neocortex, hippocampus and striatum of non-tg and α-syn tg mice;

FIG. 6B-D illustrate images of stereological estimates (dissector method) of total NeuN-positive neuronal counts in the (FIG. 6B) neocortex, (FIG. 6C) hippocampus (CA3), and (FIG. 6D) striatum,

as discussed in detail in Example 1, below.

FIG. 7A-D illustrate that systemic delivery of the siαSyn conjugated to C2-9r or ApoB¹¹ peptides reduces astrocytosis in the brain of α-syn tg mice, wherein 3-month-old α-syn tg and non-tg mice received a repeat i.p. injections of α-syn siRNA or scrambled siRNA conjugated to either C2-9r, ApoB¹¹ and were sacrificed 2 weeks later for analysis:

FIG. 7A panels graphically illustrate immunocytochemical analysis with antibodies against the astrocyte marker, GFAP in brain sections from the neocortex, hippocampus and striatum of non-tg and α-syn tg mice, and image analysis of levels of expressed as corrected optical density for GFAP immunoreactivity in the (FIG. 7B) neocortex, (FIG. 7C) hippocampus (CA3), and (FIG. 7D) striatum,

as discussed in detail in Example 1, below.

FIG. 8 illustrates an agarose gel showing that the ApoB¹¹ peptide binds to a 2′-OMe ASO in vitro to form a conjugate, where the 2′-OMe ASO targeted against ASyn was incubated with different molar ratios of the ApoB11 peptide for 30 minutes at room temperature, and complexed oligonucleotide:peptide were run through an agarose gel and uncomplexed oligonucleotide was visualized by ethidium bromide staining, as discussed in detail in Example 2, below.

FIG. 9A-D illustrates data showing that the delivery of ApoB¹¹:2′-OMe siASyn ASO reduces the accumulation of A-Syn in human neuronal cells in vitro, where human neuroblastoma cells, SH-SY5Y, were differentiated and infected with LV-ASyn or LV-Ctrl for 72 hours in differentiation media, and cells were then treated with 100 pmol siASyn (RNA), siASyn ASO (2′-OMe), siScr (scrambled RNA) conjugated to ApoB¹¹ peptide previously incubated in vitro for 30 minutes at room temperature for 48 hours:

FIG. 9A illustrates an image of representative immunohistochemistry images of coverslips stained with an antibody against ASyn (SYN211), and and LV control;

FIG. 9B illustrates a representative immunoblot from replicate SH-SY5Y neuronal cells treated as described above and probed for ASyn (SYN211) and GAPDH (loading control);

FIG. 9C graphically illustrates an image analysis of ASyn staining of SH-SY5Y ASyn stained cells represented as optical density;

FIG. 9D graphically illustrates a densitometric analysis of ASyn immunoreactive band from immunoblot at 14kDa as a ration to GAPDH. #- p<0.05 compared to untreated LV-Ctrl infected SH-SY5Y cell,

as discussed in detail in Example 2, below.

FIG. 10 A-F illustrate that delivery of ApoB¹¹:2′-OMe siα-syn to α-syn tg (Line 61) mice via intra-peritoneal injection reduces the accumulation of α-syn, where a single injection of ApoB¹¹:2-O′OMe siα-syn (50 μg) was delivered to 4-month-old α-syn tg or non-tg mice and 1 month later mice were sacrificed and analyzed for accumulation of α-syn (Syn211) (FIG. A-C) and P-syn (pSer129) (FIG. D-F), as discussed in detail in Example 2, below.

Like reference symbols in the various drawings indicate like elements.

### DETAILED DESCRIPTION

In alternative embodiments, provided are compositions, including products of manufacture and kits, and methods, for transporting nucleic acids such as oligonucleotides to and across the blood brain barrier (BBB) and into the central nervous system (CNS).

We have identified a new, alternative chimeric transport or delivery peptide for the transport of nucleotides across the blood brain barrier (BBB) based on the apolipoprotein B (apoB) protein targeted to the family of low-density lipoprotein receptors (LDL-R). In alternative embodiments, the chimeric transport or delivery peptide comprises an 11-amino acid sequence (SEQ ID NO:2) from the apoB protein (ApoB11) that, when coupled with a multi-arginine, e.g., 9-amino acids of arginine, linker, can transport oligonucleotides such as siRNAs across the BBB, e.g., to neuronal and glial cells.

In alternative embodiments, a peptide utilized in the chimeric transport or delivery peptide as provided herein comprises an endogenous peptide that would be less likely to elicit an immune response than a foreign exogenous peptide, for example, a virus-based peptide. Provided herein is evidence showing that delivery of oligonucleotides (for example, siRNAs) with the exemplary chimeric or synthetic peptide as provided herein is possible to a wide variety of cells in the CNS. Additionally, delivery of the chimeric transport or delivery peptide-oligonucleotide escapes the endoscope to the cytoplasm of the cells efficiently for action at the RNA-induced silencing complex (RISC).

In alternative embodiments, the ApoB11-comprising chimeric or synthetic peptide as provided herein can be used to deliver any form or design of nucleotides including, but limited to: natural, semi-synthetic oligonucleotides or synthetic oligonucleotides, including cDNA, mRNA, siRNA, shRNA, miRNA or gRNA.

In alternative embodiments, ApoB11-comprising chimeric or synthetic peptides as provided herein are used for the delivery of nucleic acids such as oligonucleotides to the CNS for the treatment, prevention or amelioration of a CNS disease, infection or condition, or for diagnostic purposes, for example, ApoB11-comprising chimeric or synthetic peptides as provided herein can be used for treating, preventing, ameliorating and/or diagnosing neurodegenerative diseases, CNS infections, neuro-modulatory treatments, cancers of the CNS, gene targeted editing of the CNS or anti-microbial gene delivery of the CNS. In alternative embodiments, ApoB11-comprising chimeric or synthetic peptides as provided herein are used to aid in the treatment, amelioration, prevention and/or diagnosis of a CNS disease or condition such as a neurodegenerative condition such as Alzheimer's disease, amyotrophic lateral sclerosis, neuronal ceroid lipofuscinosis (NCL) (Batten disease), Parkinson's disease, spinocerebellar ataxia (SCA) frontotemporal lobar degeneration (Pick's disease), a prion disease (Creutzfeldt-Jakob Disease) and Huntington's disease.

We have identified an alternative peptide for the transport of nucleotides across the BBB based on the apolipoprotein B (apoB) protein targeted to the family of low-density lipoprotein receptors (LDL-R). We used an 11-amino acid sequence (SEQ ID NO:2) from the apoB protein (ApoB¹¹) that, when coupled with a 9-amino acid arginine linker, can transport siRNAs across the BBB to neuronal and glial cells.

To examine the effectiveness of this peptide mediated oligonucleotide delivery system, we delivered a siRNA targeted to the α-synuclein gene in a mouse model of Parkinson's disease. We found α-syn siRNA co-localized to neurons and glial cells and subsequent reduction in α-synuclein protein accumulation in the neurons of the mouse across the whole brain. Furthermore, we observed increased neuronal numbers and decreased astrogliosis following delivery of the ApoB¹¹-si-αsyn compared to controls. Thus, we have identified an alternative delivery route for siRNA molecules that targeted an alternative receptor, thus allowing for the delivery of multiple and simultaneous nucleic acid-based therapeutics.

In alternative embodiments, compositions and methods as provided herein are used to treat, ameliorate or prevent conditions and diseases of the central nervous system (CNS), including those which arise from the accumulation of a protein such as α-synuclein in Parkinson's Disease (PD) or beta-amyloid (Aβ) in Alzheimer's disease (AD). In alternative embodiments, compositions and methods as provided herein have the ability to regulate the expression at the gene transcription level, thus being beneficial for reducing the accumulation of these proteins or regulating expression levels of other genes in the CNS.

In alternative embodiments, compositions and methods as provided herein are used to deliver short interfering RNA molecules (siRNA) to the CNS, and because the payload siRNA can bind specifically to target RNAs and cause them to be (deliver them for) degradation, the compositions and methods as provided herein are therapeutically very effective. Administration of siRNA has shown promise as a therapeutic in vitro and in mouse models of PD and AD and other neurological disorders; and in alternative embodiments the compositions and methods as provided herein overcome problems that arose when delivery of the siRNA to the CNS in vivo was been achieved primarily through intra-cranial stereotaxic injection.

In alternative embodiments, compositions and methods as provided herein can deliver nucleic acids across the BBB, which controls the passage of substances from the blood into the CNS.

In alternative embodiments, compositions and methods as provided herein use a minimal apolipoprotein B (ApoB) receptor-binding domain (SEQ ID NO:2). Binding to the receptor on the endothelial cell by the chimeric or synthetic peptide is still sufficient to trigger endocytosis and transcytosis to the neuronal side. While the invention is not limited by any particular mechanism of action, this BBB transcytosis can occur by non-specific “sticking” to apolipoproteins in the serum that then themselves bind the LDL-receptor at the BBB and trancytose the whole complex to the neuronal side.

We have identified an alternative peptide for the transport of nucleotides across the BBB based on the apolipoprotein B (apoB) protein targeted to the family of low-density lipoprotein receptors (LDL-R). Described herein is an 11 amino acid sequence (SEQ ID NO:2) from the apoB protein that, coupled with a 9 amino acid arginine linker can transport siRNAs across the BBB to neuronal and glial cells with the equal efficiency to the RGD peptide. Thus, we have identified an alternative delivery route for siRNA molecules, or any nucleic acid, that targets an alternative saturable receptor allowing the delivery of multiple, simultaneous therapeutics.

In alternative embodiments, compositions and methods as provided herein can have multiple targets for the treatment of a disease of the CNS. For example, in alternative embodiments, compositions and methods as provided herein treat or ameliorate Parkinson's disease by, for example, delivery of siRNAs for reducing α-synuclein expression, and this treatment can be couples with the delivery of antibodies for clearance of accumulated α-synuclein and compounds to prevent the aggregation of α-synuclein. Since the receptors for targeted transport across the BBB are saturable, multiple methods of transport are used, as they are necessary for the delivery of more than one therapy simultaneously.

In alternative embodiments, compositions and methods as provided herein comprise use of a ribonucleotide backbone of the RNA molecule that has been converted to a modified anti-sense oligonucleotide (ASO) such as 2′-O-(2-MethoOxyEthyl)-oligoribonucloeotides (2′-MOE) or 2′-O-Methyl-RNA (2′-OMe). ASO backbone oligonucleotides provide the advantage of increased nuclease resistance, increased half-life and increased affinity toward the mRNA target compared to the ribonucleotide backbone. This results in significantly fewer injections, lower dosages and increased effectiveness of the anti-sense oligonucleotides.

### Mimetics and Peptidomimetics

In alternative embodiments, the first peptide comprises an amino acid sequence as set forth in SEQ ID NO:2, or a mimetic or peptidomimetic thereof. In alternative embodiments, the second peptide comprises at least 2, 3, 4, 5, 6, 7, 8, 9 or 10 or more glycines, or a flexible linker, and optionally the flexible linker comprises amino acids or mimetics or peptidomimetics thereof. In alternative embodiments, the positively charged third peptide comprises at least 2, 3, 4, 5, 6, 7, 8, 9, 10, 11, 12, 13 or 14 or more positively charged amino acids or mimetics or peptidomimetics thereof, wherein optionally the positively charged amino acids comprise an arginine or a mimetic or peptidomimetic thereof, and optionally the third peptide comprises nine arginines or mimetics or peptidomimetics thereof, and optionally the chimeric or synthetic peptide comprises or consists of: RLTRKRGLKLAGGGGGRRRRRRRRR (SEQ ID NO:3) or a mimetic or peptidomimetic thereof.

In alternative embodiments, peptides and polypeptides as provided herein, or peptides and polypeptides used in methods as provided herein, include all mimetic and peptidomimetic forms. The terms “mimetic” and “peptidomimetic” refer to a synthetic chemical compound which has substantially the same structural and/or functional characteristics of a peptide or polypeptides as provided herein or as used in methods as provided herein. The mimetic or peptidomimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's or peptidomimetic's structure and/or activity. Routine experimentation can determine whether a mimetic or peptidomimetic is within the scope of the invention, i.e., that its structure and/or function is not substantially altered.

Polypeptide mimetic or peptidomimetic compositions as provided herein or used in methods as provided herein can contain any combination of non-natural structural components. In alternative aspect, mimetic or peptidomimetic forms include one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a mimetic or peptidomimetic as provided herein can have some of its residues joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, NY).

Peptides and polypeptides as provided herein, or peptides and polypeptides used in methods as provided herein, also can be contain all or some non-natural residues in place of naturally occurring amino acid residues. Non-natural residues are well described in the scientific and patent literature; exemplary non-natural compositions used as mimetics of natural amino acid residues in peptides and polypeptides as provided herein, or peptides and polypeptides used in methods as provided herein, are described below.

Mimetics of aromatic amino acids can be generated by replacing by, e.g., D- or L- naphylalanine; D- or L- phenylglycine; D- or L-2 thieneylalanine; D- or L-1, -2, 3-, or 4- pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalanine; D- or L-p-biphenylphenylalanine; D- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings. Mimetics of acidic amino acids can be generated by substitution by, e.g., non-carboxylate amino acids while maintaining a negative charge; (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholinyl-(4-ethyl) carbodiimide or 1-ethyl-3(4-azonia- 4,4-dimetholpentyl) carbodiimide. Aspartyl or glutamyl can also be converted to asparaginyl and glutaminyl residues by reaction with ammonium ions. Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, in one aspect under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylic acid, 3- or 4- hydroxy proline, dehydroproline, 3- or 4-methylproline, or 3,3,-dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A peptide or a polypeptide as provided herein, or a peptide or a polypeptide used in methods as provided herein, can also be have residues replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D- amino acid, but also can be referred to as the R- or S-form.

### Formulations and Treatment Regimens

In alternative embodiments, provided are sterile formulations, for example, sterile saline formulations, comprising a chimeric or synthetic peptide as provided herein, and optionally also comprising a nucleic acid, or positively charged molecule, payload for delivery into a cell, e.g., for delivery in vivo. In alternative embodiments, the chimeric or synthetic peptide is formulated as a sterile injectable or inhalable formulation, and optionally the chimeric or synthetic peptide is administered to the individual in need thereof by intravenous (IV), intra-peritoneal (IP), intramuscular (IM) or intrathecal injection, or by inhalation.

In alternative embodiments, the dosage of a chimeric or synthetic peptide optionally also comprising a nucleic acid payload comprises administration: daily (e.g., once a day, BID, or TID), weekly, monthly, 1 time every 2, 3, 4, 5, 6, or more months, and/or annually, the actual dosage regimen determined by clinical assessment of the patient (e.g., the severity or rate of progression, or regression, of the CNS disease, infection or condition) and responsiveness of patient to treatments as provided herein.

In alternative embodiments, the individual dosage quantity is: between about 0.1mg/kg to about 100 mg/kg protein. In alternative embodiments, formulations as provided herein are administered as single bolus, or as an infusion diluted in an appropriate solution or excipient, e.g., diluted in sterile saline or phosphate buffered saline (PBS).

Products of manufacture and Kits

In alternative embodiments, provided are products of manufacture and kits for practicing methods as provided herein, including compositions as provided herein, for example, including peptides and polypeptides as provided herein, or peptides and polypeptides used in methods as provided herein, and their nucleic acid or positively charged payloads. In alternative embodiments, provided are products of manufacture and kits optionally comprising instructions for practicing methods as provided herein.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR - Basics: From Background to Bench, First Edition, Springer Verlag, Germany.

### Example 1

### Peptide-Mediated Delivery of Oligonucleotides to the CNS Across the Blood-Brain Barrier

This example demonstrates that methods and compositions as provided herein can be used for the treatment, prevention or amelioration of a CNS disease, infection or condition, or for diagnostic purposes.

In this study, we showed that the 11-amino acid peptide derived from the apolipoprotein B fused to the 9-amino acid Arginine tail (ApoB11) could bind to and transport and RNA molecule across the BBB for delivery to neurons and astrocytes of the CNS following intra-peritoneal (i.p.) delivery. This peptide:siRNA delivery method was examined in the context of A-synuclein accumulation with a mouse model of Parkinson's/Lewy Body disease expressing A-synuclein. Delivery of the siRNA effectively reduced accumulation of A-synuclein and improved neuronal and astrocytic numbers in the CNS of the model.

### Results:

11-Amino Acid Peptide from Apolipoprotein B Delivers an RNA to Neurons In Vitro

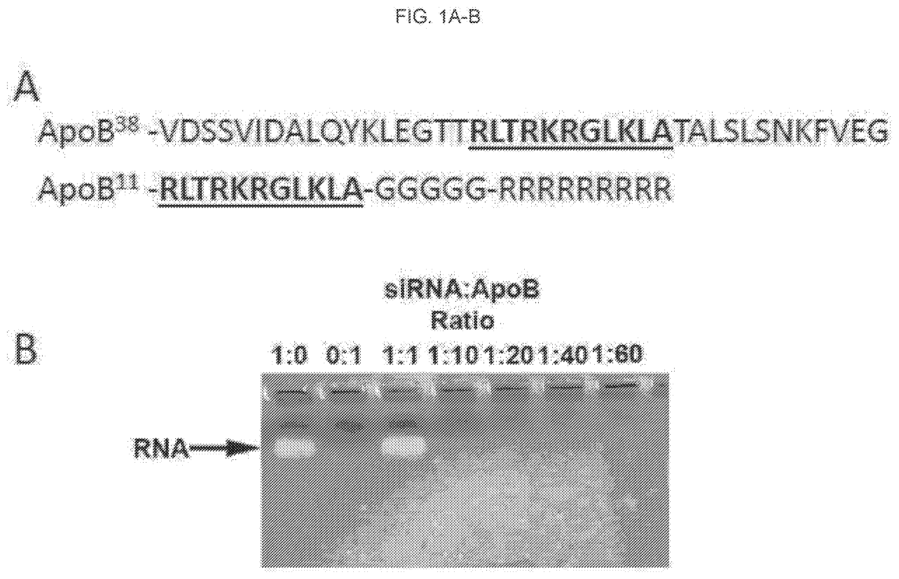

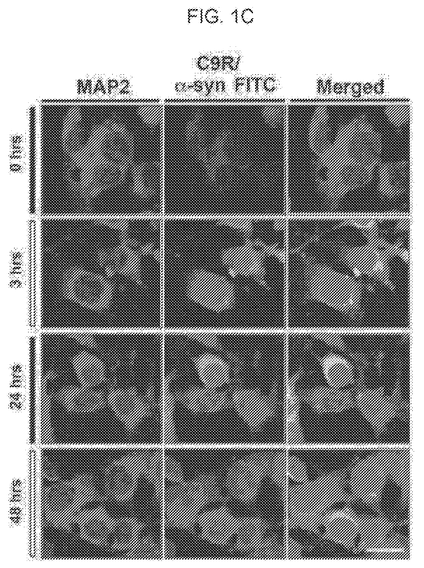

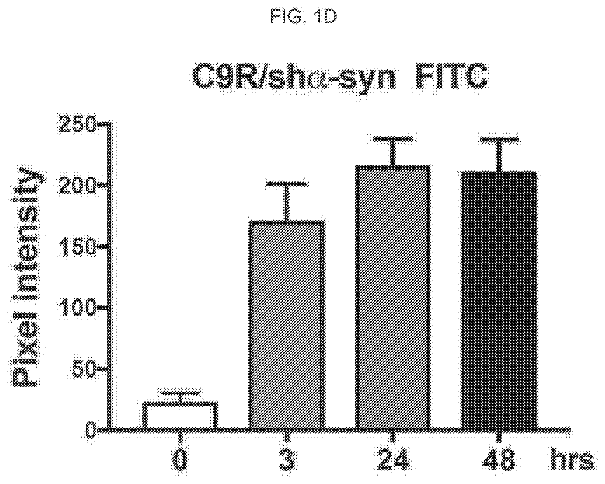

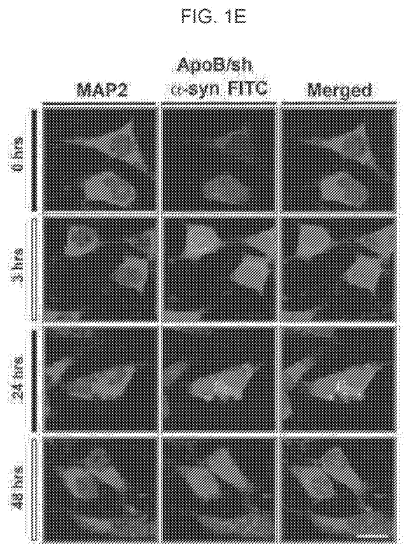

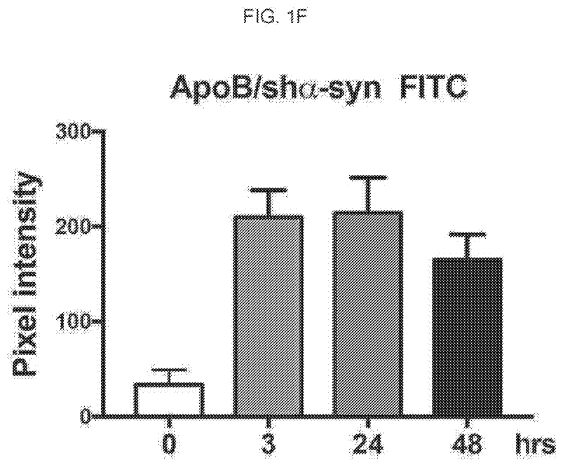

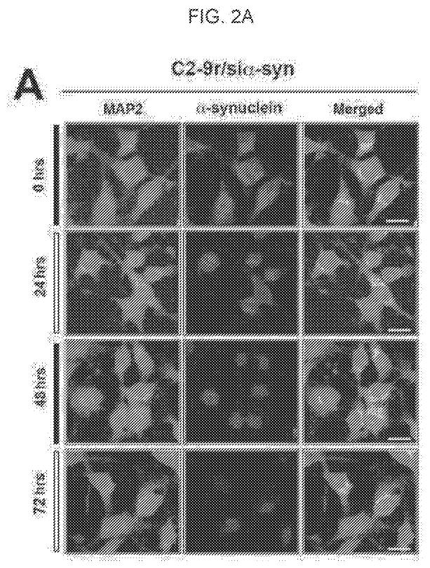

We have previously shown delivery of protein and peptides by receptor mediated transcytosis across the blood-brain barrier (BBB) with the low-density lipoprotein receptor (LDL-R) using a 38-amino acid receptor binding domain from apolipoprotein B (apoB³⁸) (SEQ ID NO:1) (see references 4 to 9). Based on literature review (see references 10, 11) we identified an 11-amino acid core domain that represented the putative minimal receptor binding domain (apoB¹¹) (SEQ ID NO:2 is the apoB¹¹ sequence/motif RLTRKRGLKLA; and, RLTRKRGLKLAGGGGGRRRRRRRRR is SEQ ID NO:3) (FIG. 1A). For delivery of oligonucleotides, we added 5 Gly (G) and 9 Arg (R) amino acids to the C-terminus (see reference 12). The glycine residues act as flexible anchor and the 9 arginine residues are a positively charged tail for binding negatively charged nucleotides (FIG. 1A).

To determine the ratio between the ApoB¹¹ peptide and RNA that leads to complete complex formation, 100 nM of siRNA was incubated with increasing molar ratios of ApoB¹¹ and run on an agarose gel. Excess RNA that was not completely bound to and charge neutralized by the 9 arginine residues of the ApoB¹¹ peptide would enter the gel and be visible by ethidium bromide staining. At a ratio of 1:0 and 1:1, unbound RNA is observable in the gel; however, at a ratio of 1:10 (0.1 μM siRNA: 1 μM ApoB¹¹), there is no free RNA to enter the gel suggesting that all the charged RNA is neutralized by the arginine residues of the ApoB¹¹ vector (FIG. 1B). For future applications, we used a ratio of 1:10 when incubating the siRNA and ApoB¹¹ vector prior to applications.

To test the ability of the ApoB¹¹ vector to deliver siRNA to neuronal cells, we incubated a fluorescein isothiocyanate (FITC) labeled siRNA with the ApoB¹¹ vector at a molar ratio of 1:10 in vitro and then added the vector to differentiated N2A neuronal cells (FIG. 1E). Shortly after incubation with the ApoB¹¹/FITC-siRNA complex, FITC label was observed throughout the cytoplasm in the neuronal cells beginning at 3 hours post-incubation. FITC signal appeared to become more punctate by 24 hours and at 48 hours was beginning to diminish indicating a degradation of either the FITC signal or the siRNA (FIG. 1F).

In order to compare the ApoB¹¹ peptide to the recently published BBB transport peptide derived from the Rabies G envelope protein (C2-9r), the C2-9r peptide was incubated with the FITC-siRNA at a ratio of 1:40 as previously published (see reference 12). C2-9r/FITC-siRNA was incubated with N2A neuronal cells and FITC label was observed at 3 hours post-incubation (FIG. 1C). FITC label increased at 24 hours and continued to 48 hours (FIG. 1D).

Alpha synuclein (α-syn) is a 140 amino acid synaptic protein (see references 13, 14) that plays a role as a scaffolding protein in vesicle trafficking, dopamine release and synaptic remodeling (see reference 15). Misfolded, aggregated α-syn has been implicated in neurological disorders with parkinsonism including Parkinson's disease (PD), Dementia with Lewy bodies (DLB) and Multiple Systems Atrophy (MSA) (Reviewed in reference 16). Given the neuronal toxicity of α-syn accumulation, therapeutic strategies for synucleinopathies might include reducing expression of the mRNA through siRNA.

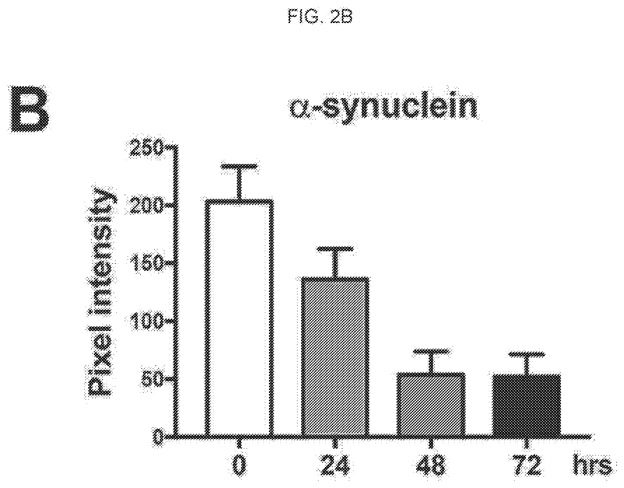

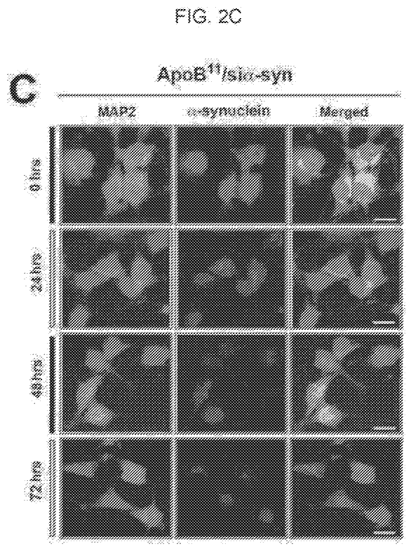

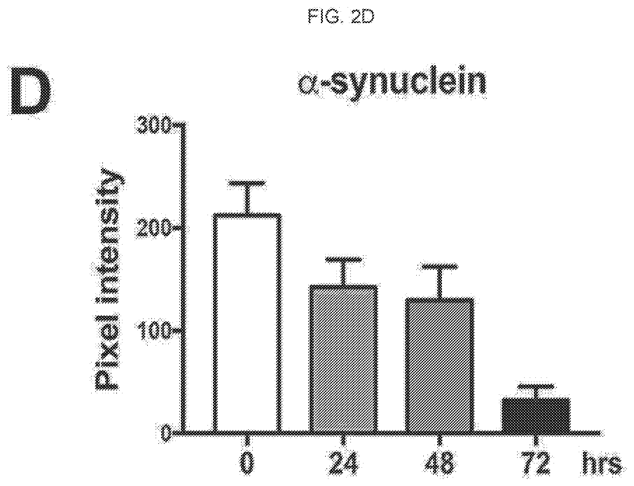

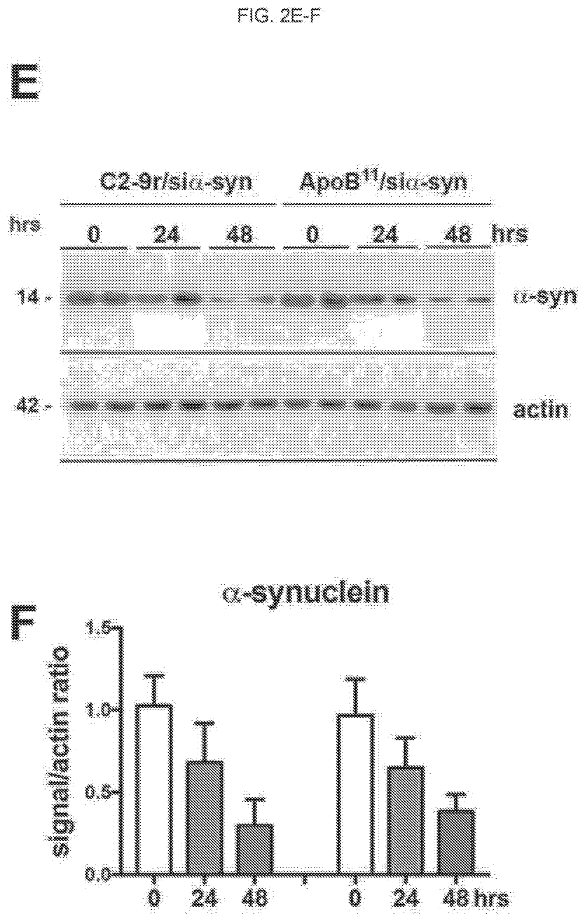

To determine if the ApoB¹¹ vector could deliver an siRNA against α-syn. The ApoB¹¹ vector was incubated with the α-syn siRNA at a molar ratio of 1:10 in vitro and then added to the N2A neuronal cells. Treatment with the ApoB¹¹-siαSyn reduced the accumulation of α-synuclein in a time dependent manner with reduced accumulation observed as early as 24 hours after treatment and nearly complete elimination of α-synuclein Immunohistochemical signal by 72 hours after treatment with the ApoB¹¹-siαSyn (FIG. 2C,D). Similar results were observed following treated with N2A cells with the C2-9r-siαSyn (FIG. 2A,B).

This was corroborated in a similar experiment where total protein was extracted and α-syn examined by immunoblot (FIG. 2E,F). By 48 hours post-treatment, α-synuclein accumulation was approximately 25% (C2-9r); 35% (ApoB¹¹) of control levels (FIG. 2F).

ApoB¹¹ and C2-9r Vectors can Deliver siRNA to the Brain

We have previously delivered peptides and proteins across the blood-brain barrier following intra-peritoneal injections when they are fused to a 38-amino acid LDL-R binding domain of ApoB. Preliminary experiments performed with fluorescein isothiocyanate (FITC) labeled siRNA conjugated to either ApoB¹¹ or C2-9r delivered to the mouse by either intra-venous or intra-peritoneal injection resulted in similar distribution to the CNS (data not shown) so for future experiments, intra-peritoneal delivery was chosen for the route of administration.

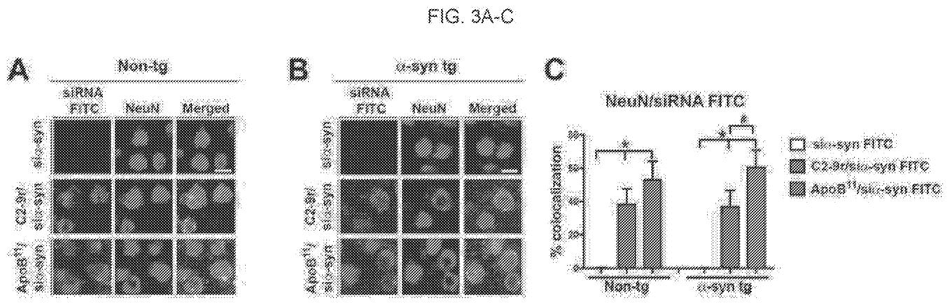

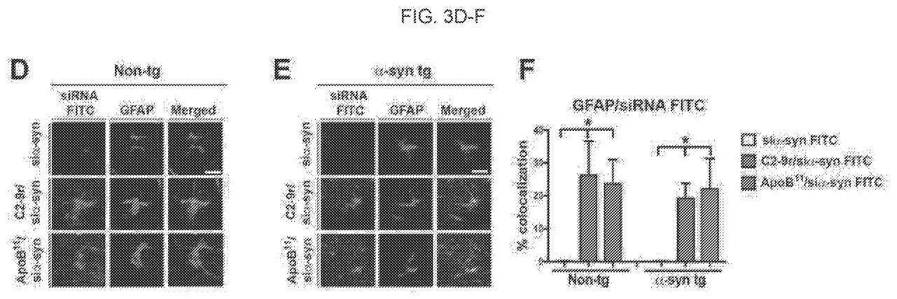

To determine if the peptides could transport siRNA across the BBB following i.p. delivery, mice received FITC labeled siαSyn conjugated to either ApoB¹¹ or C2-9r peptides. Four hours after injection, ApoB¹¹ delivered siαSyn RNA to neuronal cells as shown by co-labeling with the neuronal marker, NeuN (FIG. 3A,B). Labeling was primarily peri-nuclear in punctate patterns but was also nuclear (FIG. 3A,B). Interestingly, the ApoB¹¹ peptide delivered significantly greater FITC labeled siRNA to NeuN positive neurons than did the C2-9r peptide in α-syn tg mice (FIG. 3C). Additionally, FITC labeled siαSyn RNA co-labeled with the astrocyte marker, GFAP in the cell body but not in the processes (FIG. 3D,E). This was similar to delivery observed with proteins fused to the larger ApoB³⁸ peptide (see references 6 to8). Similarly, the C2:9r:siαSyn(FITC) delivered by i.p. injection showed neuronal and astrocyte localization in non-tg and α-syn tg mice with co-localization of the siRNA (FITC) and NeuN and GFAP markers respectively (FIG. 3).

Delivery of FITC labeled siαSyn conjugated to either ApoB¹¹ or C2-9r reduced α-syn protein levels cells of the cortex (FIG. 4A,B). The FITC label of the siαSyn co-localized with αSyn expressing cells (FIG. 4A,B) indicating that the ApoB¹¹ and C2-9r peptides are capable of delivering the siRNA to the αSyn expressing cells and that the siRNA is able to then reach the appropriate sub-cellular target.

Peptide Mediated Delivery of siRNA Prevents α-Synuclein Accumulation

To determine if the ApoB¹¹ vector could be used to silence gene expression in the brain following systemic i.p. delivery, we used the α-synuclein tg mouse model of Parkinson's/Dementia with Lewy Body disease. 3-month-old α-syn tg and non-tg mice received either the siαSyn or si-scrambled RNA conjugated to ApoB¹¹ or C2-9r peptides. The si-scrambled was a scrambled RNA with the same ratio of nucleotides as the siαSyn and incubated at the same ratio of 1:10 with ApoB¹¹ or 1:40 with C2-9r in vitro prior to in vivo delivery. Mice received intra-peritoneal injections of 50 μg siRNA every 3 days for 2 weeks and 3 days after the last injection were sacrificed. Brains were removed with half flash frozen for protein analysis and half fixed in 4% paraformaldehyde for vibratome sections for immunohistochemistry.

In contrast to treatment with the ApoB¹¹-si-scrambled where accumulation of α-synuclein can be observed throughout the neocortex and hippocampus (CA3) as well as the striatum of the transgenic mouse, treatment with the ApoB¹¹-siαSyn reduced overall accumulation of α-synuclein throughout the whole brain (FIG. 5A-D). Specifically, α-synuclein was reduced in neuronal cells in the cortex, hippocampus CA3 region and the neuropil of the striatum. Reduction of α-syn by immunohistochemistry was approximately 50% in all regions examined (FIG. 5B-D). Immunoblot analysis of the whole brain confirmed the reduction of α-synuclein (65%) in ApoB¹¹-siαSyn treated mice (FIG. 5E,F). Similar results were obtained from delivery of siαSyn by the C2-9r peptide (50%).

We have previously reported that the α-syn tg model displays loss of neurons and astrogliosis in the CA3 region of the hippocampus and neocortex (see references 17, 18). As a control, we evaluated the striatum which shows little to no change in the α-syn tg mouse (see references 26, 27). To evaluate the effects of modulating α-syn expression in the α-syn tg mouse, levels of NeuN and GFAP were analyzed. Compared to non-tg mice, α-syn tg mice displayed decreased NeuN neurons in the neocortex and CA3 region of the hippocampus as previously reported (see reference 19) (FIG. 6A-D). Treatment of α-syn tg mice with the ApoB¹¹-siαSyn restored levels NeuN cell counts in both the neocortex and CA3 comparable to non-tg mice (FIG. 6A-D).

α-syn tg mice treated with the si-scrambled displayed increased GFAP immunoreactivity in the neocortex and the CA3 region of the hippocampus (FIG. 7A-D). Treatment of α-syn tg mice with the siαSyn-ApoB¹¹ reduced levels of GFAP immunoreactivity to levels observed in non-tg mice (FIG. 7A-D). Similarly, α-syn tg mice treated with siαSyn conjugated to C2-9r showed increased NeuN and reduced GFAP staining in the neocortex and hippocampus following intra-peritoneal delivery (FIGS. 6 and 7).

In summary, this data shows the delivery of an siRNA against α-synuclein delivered across the BBB by conjugation with an 11-amino acid peptide derived from Apolipoprotein B (ApoB¹¹) following intra-venous injection. Similarly, the same siRNA is can be transported across the BBB to the neurons and astrocytes using the C2-9r peptide derived from the Rabies G envelope protein. Delivery of the siRNA functions to reduce the accumulation of α-synuclein protein in an animal model of PD/DLB.

### Discussion:

In this study, we showed that the 11-amino acid peptide (SEQ ID NO:2) derived from the apolipoprotein B fused to the 9-amino acid Arginine tail (ApoB¹¹) could bind to and transport and RNA molecule across the BBB for delivery to neurons and astrocytes of the CNS following intra-peritoneal delivery. This peptide:siRNA delivery method was examined in the context of α-synuclein accumulation with a mouse model of Parkinson's/Lewy Body disease expressing α-synuclein. Delivery of the siRNA effectively reduced accumulation of α-synuclein and improved neuronal and astrocytic numbers in the CNS of the model. The ApoB¹¹ peptide could be used to deliver any form or design of nucleotides including, but limited to: cDNA, mRNA, siRNA, shRNA, miRNA, gRNA or synthetic nucleotide sequences. Delivery of oligonucleotides to the CNS could be beneficial for neurodegenerative diseases, neuro-modulatory treatments, cancers of the CNS, gene targeted editing of the CNS or anti-microbial gene delivery of the CNS.

Delivery of siRNA by either the ApoB¹¹ or C2-9r peptide to the CNS occurred primarily to neurons and only to a lesser extent the astrocytes as measured by co-labeling studies with NeuN and GFAP respectively. We previously showed with the larger ApoB³⁸ peptide fused to the protease, neurosin, that uptake occurred primarily with neurons and to a lesser extent astrocytes, microglia and oligodendrocytes [7]. This pattern closely resembled the pattern of LDL-R expression levels on these cells as the peptide relies on this receptor for endocytosis into the cell [7]. Following endocytosis, the siRNA must escape the endosome as a naked oligonucleotide to function as an inhibitor molecule. The process of this escape mechanism is unclear; however, previous studies have shown other proteins and antibodies have escaped the endosome following endocytosis with the ApoB³⁸ peptide suggesting a mechanism must exist [4-7, 9]. It's possible that binding to HDL or LDL molecules through the ApoB peptide may facilitate escape of the endosome via SR-BI receptor, thus bypassing the lysosome fusion [20, 21]. This would allow escape of the peptide:RNA complex to the cytoplasm where it could access the mRNA for RISC mediated endonuclease digestion [22].

Although the t_{1/2} of the siRNA wasn't examined in this study, previous studies have shown the half-life of unprotected RNA in the serum to be approximately 2 hours [12, 23] and in the rodent following intravenous delivery of 6 minutes [24]. Once inside the cell, the siRNA has a long half-life and has shown knockdown of the target gene for as long as 3 days in dividing cells and 3-4 weeks in non-dividing cells [23, 25]. In order to increase the effectiveness and therapeutic potential of siRNAs for CNS delivery, longer t_{1/2} in the serum would be beneficial. Introduction of 2′-fluoro or 2′-O′methyl sugar modifications function to increase endonuclease resistance and stabilizing siRNA duplexes. In fact, 2′-F modified siRNAs have an increased serum t_{1/2} of approximately 24 hours [23]. Currently, 2′-F modified siRNAs are approved for clinical use (Fomivirsen and Macugen) [26]. Other modifications that could be considered include morpholinos, peptide nucleic acids and phosphorthioate sugars [27].

The Apolipoprotein B protein binds to the LDL receptor at the surface of the endothelial cells on the BBB inducing endocytosis and transytosis to the CNS side where it is released for binding to receptors on neurons and astrocytes. Similarly, we believe the ApoB¹¹ peptide binds to the LDL-R and transports siRNA to the CNS. In contrast, the RGD peptide binds to the nicotinic acetylcholine receptor (nAch) [28] on neurons for uptake, although the receptor used for binding and transport across the blood-brain barrier is not known as nAch and other known RGD binding receptors are not expressed on BBB endothelial model cells such as bEnd.3 or BCECs [29]. However, other atypical nAch a-subunits have been detected by RT-PCR in rat arterial endothelial cells possibly explaining the uptake and transport mechanism at the BBB [30, 31]. These alternate receptors may also be responsible for the uptake by astrocytes. In fact, the α7 nAchR has been detected in cultured neonatal rat microglial cells [32]. Recent studies have shown and upregulation of nAchR in the caudate nucleus of Parkinson's patients with a concomitant downregulation in the putamen, thus potentially complicating the targeting of the C2-9r directed siRNA for Parkinson's disease [33]. It remains to be seen what role each of these peptides may play in therapeutic interventions for different targets.

Diseases of the CNS typically have multiple targets for treatment. For instance, Parkinson's disease targets currently include the delivery of siRNAs for reducing α-synuclein expression, antibodies for clearance of accumulated α-synuclein and compounds to prevent the aggregation of α-synuclein. Since the transport with receptors targeted for transport across the BBB are saturable [34, 35], identification of multiple methods of transport is necessary for the delivery of more than one therapy simultaneously. Starzyk et al showed that the transferrin receptor could be used for transport of protein across the BBB [36] and subsequently, the IGF-R was used to transport protein [37, 38]. These receptors work similarly to the LDLR family of receptors in that they (a) bind protein on the basal/blood side, (b) transport by transytosis to the neuronal side, and (c) release the protein on the neuronal side of the BBB [39]. Targeting different receptor pathways for different therapeutics will prevent the possibility of overwhelming the transport kinetics at the BBB thus allowing simultaneous delivery of multiple therapeutic options.

In summary, we have identified a 11-amino acid LDL-R binding peptide that can transport oligonucleotides across the BBB for therapeutic options of the CNS. The ApoB¹¹ peptide, along with the C2-9r peptide, represent two potential approaches for the delivery of siRNAs or other oligonucleotides to the CNS for therapies. It remains to be seen if the 11-amino acid ApoB peptide can function to transport larger proteins with the same efficiency as the previously published 38 amino acid ApoB³⁸ peptide.

### Methods:

### Generation of ApoB¹¹

The ApoB¹¹ peptide (NH₂RLTRKRGLKLAGGGGGRRRRRRRRR) (SEQ ID NO:4) (90% purity) was purchased from Karebay Biochem, Inc., and the C2-9r was provided by Omar El-Agnaf [12]. Peptides were diluted to 10 mM in PBS (Life Technologies). The siαSyn (5′GAC UUU CAA AGG CCA AGG A) (SEQ ID NO:5) corresponding to nucleotides 168-186 and si-scrambled (5′GGG CAU ACU GAG CUA ACA A) (SEQ ID NO:6) RNAs were purchased from ValueGene, Inc. and desalted. FITC labeled siαSyn RNA was labeled at 5′. Oligos were resuspended in RNAse free water (Life Technologies) at 100 mM and aliquoted. siRNA and peptide were mixed at specified ratio in PBS and incubated at room temperature for 30 minutes to allow RNA to bind to peptide.

### Neuronal Cell Lines Treatments

The mouse cholinergic cell line cell line Neuro2A (N2A) was utilized for in vitro experiments [40]. N2A cells were plated at 1×10⁵ cells/well of a 12 well dish on poly L-lysine coated coverslips in DMEM+1% FBS for 5 days to allow for differentiation [41]. Cells were treated with 100 pmol siRNA:peptide and then fixed in 4% paraformaldehyde. Immunolabeling was performed with an antibody against MAP2 (Millipore) and analysis of uptake of the FITC labeled siRNA was performed by laser scanning confocal microscopy (BioRad, MRC1024) [41].

Additionally, N2A cells were double immunolabeled with antibodies against MAP2 (Millipore) and α-syn (polyclonal Millipore). α-syn was detected with the Tyramide Red (NEN Life Sciences) whereas MAP2 was detected with FITC-tagged antibodies (Vector). Coverslips were imaged with a Zeiss 63×1.4 objective on an AXIOVERT 35™ microscope (Zeiss) with an attached MRC1024 laser scanning confocal microscope (LSCM) system (BioRad) and analyzed with Image J to determine pixel intensity [41].

### Animal Care

All experiments described were carried out in strict accordance with good animal practice according to NIH recommendations, and all procedures for animal use were approved by the Institutional Animal Care and Use Committee at the University of California at San Diego (UCSD) under protocol #S07221.

### Transgenic Mouse Lines and Injections of Lentiviral Vectors

For this study, mice over-expressing α-synuclein from the platelet-derived growth factor β (PDGF-β) promoter (Line D) were utilized [42, 43]. This model was selected because mice from this line develop intraneuronal α-synuclein aggregates distributed through the neocortex and hippocampus similar to what has been described in LBD.

To determine the effects of systemic injections of the siRNA conjugated to a BBB transport peptide, the following injections were performed: siαSyn:ApoB¹¹→α-syn tg n=4, si-scrambled:ApoB¹¹→α-syn tg n=4, siαSyn:C2-9r →α-syn tg n=4, si-scrambled:C2-9r →α-syn tg n=4, siαSyn:ApoB¹¹→non tg n=3, si-scrambled:ApoB¹¹→α-non tg n=3, siαSyn:C2-9r →α-non tg n=3, si-scrambled:C2-9r→α-non tg n=3. Mice received intra-peritoneal injections of 50 μg of siRNA conjugated to either C2-9r or ApoB¹¹ peptide twice weekly for two weeks.

To co-localize the siRNA with neuronal and astrocytic markers as well as α-synuclein accumulation, mice (n=2 per condition) received a single intra-peritoneal injection of 50 μg of 5′ FITC labeled si-α-syn conjugated to either C2-9r or ApoB¹¹ or naked and 4 hours later were sacrificed.

Following NIH guidelines for the humane treatment of animals, mice were anesthetized with chloral hydrate and flush-perfused transcardially with 0.9% saline. Brains and peripheral tissues were removed and divided sagitally. The right hemibrain was post-fixed in phosphate-buffered 4% PFA (pH 7.4) at 4° C. for 48 hours for neuropathological analysis, while the left hemibrain was snap-frozen and stored at -70° C. for subsequent protein analysis.

### Immunoblot Analysis

Hemibrains were homogenized as previously described [44, 45]. For immunoblot analysis, 20 μg of total protein per lane was loaded on 4-12% Bis-Tris SDS-PAGE gels and blotted onto polyvinylidene fluoride membranes. Membranes were probed with antibodies against full-length human α-syn (SYN211, Life Technologies). Incubation with primary antibody was followed by species-appropriate incubation with secondary antibody tagged with horseradish peroxidase (Santa Cruz Biotechnology) and visualization with enhanced chemiluminescence. Analysis of all immunoblot was performed with a Versadoc XL™ imaging apparatus (BioRad) using β-actin (Sigma) levels as a loading control.

### Immunocytochemical and Neuropathological Analyses

To determine the co-localization between fluorescein isothiocyanate (FITC)-siαSyn and cellular markers, double labeling experiments were performed as previously described (44, 45). 40 p.m-thick vibratome sections were immunolabeled with the rabbit polyclonal antibody against α-synuclein (Chemicon, affinity purified polyclonal), NeuN (neuronal marker, Millipore) or GFAP (astroglial marker, Chemicon) and the immunoreactive structures were detected with the Tyramide Signal Amplification™-Direct (Red) system (NEN Life Sciences) while the siRNA was directly visualized by the appended FITC label. Sections were imaged with a Zeiss 63X™ (N.A. 1.4) objective on an Axiovert 35™ microscope (Zeiss) with an attached MRC1024™ LSCM system (BioRad) [22].

Analysis of α-synuclein accumulation was performed in serially sectioned, free-floating, blind-coded vibratome sections from tg and non-tg mice treated with peptide conjugated siRNA. Sections were incubated overnight at 4° C. with an anti-α-synuclein antibody (affinity purified rabbit polyclonal, Chemicon) [42] followed by biotinylated secondary antibody and reacted with diaminobenzidine to determine the number of hα-synuclein immunoreactive inclusions in the neocortex, hippocampus and striatum.

To determine if the siαSyn gene transfer ameliorated the neurodegenerative alterations associated with the expression of α-syn, briefly as previously described [44], blind-coded, 40-μm thick vibratome sections from mouse brains fixed in 4% paraformaldehyde were immunolabeled with the mouse monoclonal antibodies against NeuN (neuronal marker, Millipore) or glial fibrillary acidic protein (GFAP, astroglial marker, Chemicon) [44]. After overnight incubation with the primary antibodies, sections were incubated with biotinylated secondary antibody and reacted with diaminobenzidine, transferred to SuperFrost™ slides (Fisher Scientific). All sections were processed under the same standardized conditions. For each mouse, a total of three sections each were analyzed and for each section, three fields each in the frontal cortex, hippocampus and striatum were examined. For NeuN, results were expressed as numbers of positive cells and for GFAP and Synuclein, results were expressed as corrected optical density. Optical density measurements were obtained using the ImageQuant™ software and quantification performed by correcting against background signal levels. All sections were processed simultaneously under the same conditions and experiments were performed twice in order to assess the reproducibility of results. Sections were imaged with a digital scanning microscope (Zeiss).

### Statistical Analysis

All experiments were performed blind coded and in triplicate. Values in the figures are expressed as means±SEM. To determine the statistical significance, values were compared by using the one-way ANOVA with posthoc Dunnet when comparing the scFV treated samples to LV-control treated samples. Additional comparisons were done using Tukey-Krammer or Fisher posthoc tests. The differences were considered to be significant if p values were less than 0.05.

### Figure Legends:

FIG. 1: A non-viral peptide can deliver a short RNA to neuronal cells.

(A) We have previously used a 38-amino acid peptide derived from the Apolipoprotein B protein for delivery of proteins and peptides for receptor mediated BBB transcytosis to the CNS. For this project, we used a shorter, 11 amino acid (underlined) fragment coupled to a 5 Glycine linker and a 9 Arginine positively charged tail. (B) siRNA was incubated with different molar ratios of the ApoB¹¹ peptide for 30 minutes. Complexed RNA and protein was run through an agarose gel and unconjugated RNA was observed by ethidium bromide staining. (C,E) FITC labeled siRNA were added to N2A mouse neuronal cells at a ratio of 1:10 siRNA:ApoB¹¹ or 1:40 siRNA:C2-9r and analyzed at different time points for uptake of the FITC labeled siRNA. (D,F) Coverslips were analyzed for siRNA (FITC signal) pixel intensity. Scale bar represents 15 μm.
FIG. 2: Peptide delivery of siRNA targeting α-synuclein reduced the accumulation of α-synuclein in neuronal cells in vitro. (A)

N2A neuronal cells were treated with 100 pmol siαSyn conjugated to the (A) C2-9r peptide or to the (C) ApoB¹¹ peptide and immunostained for α-synuclein and the neuronal marker MAP2. (B,D) coverslips were analyzed for α-synuclein pixel intensity. (E) Representative immunoblot from replicate from N2A cultures treated with siαSyn conjugated to the C2-9r peptide or the ApoB¹¹ peptide. (D) Densitometry analysis of the α-synuclein bands as a ratio to β-actin. Scale bar represents 10 μm.
FIG. 3: Peptide-siRNA is distributed to the brain following i.p. delivery.

Non-tg and α-syn tg mice received a single i.p. injection of fluorescein isothiocyanate (FITC) labeled α-syn siRNA conjugated to either C2-9r, ApoB¹¹ or naked. 4 hours after injection, mice were sacrificed, whole brain removed and fixed. (A,B) Sections were stained for the neuronal marker NeuN and the nuclear stain DAPI and then visualized for co-localization with the FITC labeled siRNA. (D,E) Sections were stained for the astrocytic marker GFAP and the nuclear stain DAPI and then visualized for co-localization with the FITC labeled siRNA. Analysis of % co-localization between (C) NeuN (red) and siRNA (green) or (F) GFAP (red) and siRNA (green). * indicates statistical significance p<0.05 compared to siRNA alone. # indicates statistical significance p<0.05 compared to C2-9r siRNA. Scale bar represents 25 μm.
FIG. 4: Peptide-siRNA co-localizes with and reduces the accumulation of α-synuclein in the brain following i.p. delivery.

(A) Non-tg and (B) α-syn tg mice received a single i.p. injection of FITC labeled α-syn siRNA conjugated to either C2-9r, ApoB¹¹ or naked. 4 hours after injection, mice were sacrificed, whole brain removed and fixed. Sections were stained for α-synuclein and the nuclear marker, DAPI and then visualized for co-localization with the FITC labeled siRNA. Scale bar represents 25 μm.
FIG. 5: Systemic delivery of the siαSyn conjugated to C2-9r or ApoB¹¹ peptides reduces the accumulation of α-synuclein in the brain of transgenic mice.

3-month-old α-syn tg and non-tg mice received a repeat i.p. injections of α-syn siRNA or scrambled siRNA conjugated to either C2-9r, ApoB¹¹ and were sacrificed 2 weeks later for analysis. (A) Immunocytochemical analysis with antibodies against the αsynuclein in brain sections from the neocortex, hippocampus and striatum of non-tg and α-syn tg mice. Image analysis of levels of expressed as corrected optical density for α-synuclein immunoreactivity in the (B) neocortex, (C) hippocampus (CA3), and (D) striatum. (E) Representative, immunoblot analysis of brain homogenates from non-tg and αsyn tg mice treated with α-syn siRNA or scrambled siRNA conjugated to either C2-9r, ApoB¹¹. (F) Image analysis of pixels of α-synuclein immunoreactivity analyzed as ratio to β-actin signal. * indicates statistical significance p<0.05 compared to non-tg mice. ** p<0.05 compared to C2-9r/si-scrambled. # indicates statistical significance p<0.05 compared to α-syn-tg mice treated with ApoB¹¹-si-scrambled by one-way ANOVA with post hoc Tukey-Kramer. Scale bar represents 250 μm for low magnification panels and 25 μm for higher magnification panels.
FIG. 6: Systemic delivery of the siαSyn conjugated to C2-9r or ApoB¹¹ peptides reduces neurodegeneration in the brain of α-syn tg mice.

3-month-old α-syn tg and non-tg mice received a repeat i.p. injections of α-syn siRNA or scrambled siRNA conjugated to either C2-9r, ApoB¹¹ and were sacrificed 2 weeks later for analysis. (A) Immunocytochemical analysis with antibodies against the neuronal marker, NeuN in brain sections from the neocortex, hippocampus and striatum of non-tg and α-syn tg mice. Stereological estimates (dissector method) of total NeuN-positive neuronal counts in the (B) neocortex, (C) hippocampus (CA3), and (D) striatum. * indicates statistical significance p<0.05 compared to non-tg mice. ** p<0.05 compared to C2-9r/si-scrambled. # indicates statistical significance p<0.05 compared to α-syn-tg mice treated with ApoB¹¹-si-scrambled by one-way ANOVA with post hoc Tukey-Kramer. Scale bar represents 250 μm for low magnification panels and 25 μm for higher magnification panels.
FIG. 7: Systemic delivery of the siαSyn conjugated to C2-9r or ApoB¹¹ peptides reduces astrocytosis in the brain of α-syn tg mice.

3-month-old α-syn tg and non-tg mice received a repeat i.p. injections of α-syn siRNA or scrambled siRNA conjugated to either C2-9r, ApoB¹¹ and were sacrificed 2 weeks later for analysis. (A) Immunocytochemical analysis with antibodies against the astrocyte marker, GFAP in brain sections from the neocortex, hippocampus and striatum of non-tg and α-syn tg mice. Image analysis of levels of expressed as corrected optical density for GFAP immunoreactivity in the (B) neocortex, (C) hippocampus (CA3), and (D) striatum. * indicates statistical significance p<0.05 compared to non-tg mice. ** p<0.05 compared to C2-9r/si-scrambled. # indicates statistical significance p<0.05 compared to α-syn-tg mice treated with ApoB¹¹-si-scrambled by one-way ANOVA with post hoc Tukey-Kramer. Scale bar represents 250 μm for low magnification panels and 25 μm for higher magnification panels.

### Example 2

### 2′-OMe Backbone siRNA Targeted to the Human/Mouse ASyn Sequence

We converted an established siRNA targeted to the human/mouse ASyn sequence [47] to the 2′-OMe backbone and showed that this can bind to the ApoB¹¹ peptide in vitro to form the peptide:oligonucleotide conjugate, see FIG. 8. In order to determine if the ApoB¹¹:2-OMe ASyn could reduce the accumulation of ASyn in a neuronal cell line as efficiently as the RNA sequence, we differentiated SH-SY5Y human neuroblastoma cells and infected with a lentivirus vector overexpressing human ASyn (LV-ASyn) or a control vector (LV-Ctrl). Cells were treated with the ApoB¹¹:oligonucleotide for 48 hours and then either fixed/stained for ASyn or cell lysates were collected/analyzed by immunoblot for ASyn protein. Treatment with the ApoB¹¹:siASyn (RNA backbone) or ApoB¹¹:2′-OMe siASyn had significant lower levels of ASyn accumulation in cells that were overexpressing ASyn (FIG. 9A,C). The 2′-OMe siASyn significantly reduced endogenous levels of ASyn in the SH-SY5Y cells infected with the LV-Ctrl virus as determined by immunohistochemistry where the ApoB¹¹:si-ASyn (RNA backbone) did not (FIG. 3A,C). These results were confirmed by immunoblot (FIG. 9B,D). Thus, the 2′OMe backbone was efficiently transport into cells and reduced the expression of ASyn in neurons.

Delivery of siRNAs to the brain using i.p. injection has been validated: data has been shown that ApoB¹¹ can bind to and transport RNA molecules across the BBB for delivery to neurons and astrocytes (Spencer, Trinh et al. 2019). This peptide: siRNA delivery method was examined in the context of α-syn accumulation using a mouse model of PD/DLB (D-Line, α-syn tg) expressing α-syn. Systemically-delivered ApoB¹¹/siα-syn reduced α-syn accumulation, neurodegeneration and neuroinflammation (Spencer, Trinh et al. 2019). Collectively, these data demonstrate that ApoB¹¹:siα-syn is effective in reducing the expression of α-syn and may be an effective therapeutic option.

To translate this technology for clinical delivery, we converted the siRNA sequence to an antisense oligonucleotide (ASO) 2′-O-Methyl-oligoribonucloeotides (2′-OMe) to take advantage of increased nuclease resistance, increased half-life and increased affinity toward the mRNA target compared to the ribonucleotide backbone allowing for significantly fewer injections, lower dosages and increased effectiveness (Layzer, McCaffrey et al. 2004, Chan, Lim et al. 2006).

We converted the mouse/human cross-reacting siRNA α-syn sequence to the 2′-OMe backbone. To determine the ratio between the ApoB11 peptide and 2′-OMe ASO that leads to complete complex formation, 100 nM of ASO was incubated with increasing molar ratios of ApoB 11 and run on an agarose gel (FIG. 1). Excess ASO that was not completely bound to and charge neutralized by the 9 arginine residues of the ApoB¹¹ peptide would enter the gel and be visible by ethidium bromide staining. At a ratio of 1:1 and 1:2, all unbound ASO is observable in the gel; however, at a ratio of 1:5 some of the ASO is clearly retarded by the peptide as less is visible in the gel. At a ratio of 1:10 (0.1 μM siRNA: 1 μM ApoB11), there is no free ASO to enter the gel suggesting that all the charged ASO is neutralized by the arginine residues of the ApoB 11 vector, see FIG. 8.

In order to determine whether ApoB¹¹:2-OMe siα-syn can reduce accumulation of α-syn in a neuronal cell line as efficiently as the RNA backbone (siRNA α-syn), we infected differentiated SH-SY5Y human neuroblastoma cells with a lentivirus vector overexpressing human α-syn (LV-α-syn) or control vector (LV-Ctrl). Cells were then treated with ApoB¹¹:si α-syn (alphα-syn) or ApoB¹¹:si scrambled for 48 hours and then either fixed and immunostained for α-syn or lysed for immunoblot analysis of α-syn protein. We found that both untreated cells and cells treated with scrambled siRNA showed high levels of endogenous α-syn, and even further increased α-syn levels following infection with the LV-α-syn virus (FIG. 2A,C). Treatment with ApoB¹¹:si α-syn (RNA backbone) or ApoB¹¹:2′-OMe siα-syn significantly reduced levels of α-syn protein in α-syn overexpressing cells (FIG. 2A,C). 2′-OMe si α-syn significantly reduced endogenous levels of α-syn in SH-SY5Y cells infected with the LV-Ctrl virus whereas ApoB¹¹:si α-syn (RNA backbone) did not (FIG. 9A, FIG. 9C). These results were confirmed with immunoblot (FIG. 9B, FIG. 9D). Collectively, these data demonstrate that 2′-OMe siα-syn ASO is as effective at reducing expression and accumulation of α-syn as the original ribonucleotide siα-syn in an in vitro model of neuronal α-syn expression (FIG. 9A-D).

To determine if the ApoB¹¹:2′-OMe siα-syn could reduce accumulation of α-syn in vivo, we delivered the peptide:oligonucleotide conjugate (5 μg oligonucleotide) to either α-syn tg (Line 61, Thy-1 α-syn) or non tg mice by i.p. delivery and then sacrificed the mice one month later for examination of α-syn accumulation by immunohistochemistry. ApoB¹¹:2′-OMe si scrambled (5 μg) was used as a control. Mice receiving 2′-OMe si scrambled oligonucleotide showed high levels of α-syn staining (Syn211) in the cortex and hippocampus, whereas, mice that received ApoB¹¹:2′-OMe siα-syn had significantly less α-syn containing cells (FIG. 10A-C). Similarly, mice that received ApoB¹¹:2′-OMe siα-syn showed significantly fewer positive phospho-α-syn positive cells in the hippocampal CA1 region compared to mice that received the si scrambled oligonucleotide (FIG. 10D-F). Thus, a single i.p. injection of the ApoB¹¹:2′-OMe siα-syn was sufficient to reduce accumulation of α-syn 1 month after injection in an α-syn tg mouse.

In summary, this data showed that delivery of an ASO against α-synuclein delivered to neurons using an exemplary chimeric or synthetic peptide as provided herein reduced the expression of the targeted gene. The ApoB¹¹ peptide can be used to deliver any form or design of nucleotides including, but not limited to: cDNA, mRNA, siRNA, shRNA, miRNA, gRNA or synthetic nucleotide sequences. The addition of the arginine or similar charged tail allows binding of the nucleotide sequence to the ApoB¹¹ peptide leading to transport across the BBB via the LDL-R. Applications can include treatment of neurodegenerative diseases, neuro-modulatory treatments, cancers of the CNS, gene targeted editing of the CNS or anti-microbial gene delivery to the CNS.

FIG. 1A illustrates the characteristics of a non-viral peptide derived from ApoB that delivers a short RNA targeting α-syn to neuronal cells, including diagrammatic illustration of the original 38-amino acid peptide derived from the Apolipoprotein B protein for delivery of proteins and peptides for receptor mediated BBB transcytosis to the CNS and the novel shorter, 11 amino acid (underlined) fragment coupled to a 5 Glycine linker and a 9 Arginine positively charged tail utilized for delivering siRNA into neuronal cells.

FIG. 8 illustrates that the ApoB¹¹ peptide binds to a 2′-OMe ASO in vitro to form a conjugate. The 2′-OMe ASO targeted against ASyn was incubated with different molar ratios of the ApoB¹¹ peptide for 30 minutes at room temperature. Complexed oligonucleotide:peptide were run through an agarose gel and uncomplexed oligonucleotide was visualized by ethidium bromide staining.

FIG. 9A-D illustrates data showing that the delivery of ApoB¹¹:2′-OMe siASyn ASO reduces the accumulation of A-Syn in human neuronal cells in vitro. Human neuroblastoma cells, SH-SY5Y, were differentiated and infected with LV-ASyn or LV-Ctrl for 72 hours in differentiation media. Cells were then treated with 100 pmol siASyn (RNA), siASyn ASO (2′-OMe), siScr (scrambled RNA) conjugated to ApoB¹¹ peptide previously incubated in vitro for 30 minutes at room temperature for 48 hours.

FIG. 9A illustrates a representative immunohistochemistry images of coverslips stained with an antibody against ASyn (SYN211).

FIG. 9B illustrates a representative immunoblot from replicate SH-SY5Y neuronal cells treated as described above and probed for ASyn (SYN211) and GAPDH (loading control).

FIG. 9C illustrates an image analysis of ASyn staining of SH-SY5Y ASyn stained cells represented as optical density.

FIG. 9D illustrates a densitometric analysis of ASyn immunoreactive band from immunoblot at 14kDa as a ration to GAPDH. #-p<0.05 compared to untreated LV-Ctrl infected SH-SY5Y cells. *-p<0.05 compared untreated, LV-ASyn infected SH-SY5Y infected cells. One-way ANOVA with poshoc Tukey-Kramer.

FIG. 10 A-F illustrate that delivery of ApoB¹¹:2′-OMe siα-syn to α-syn tg (Line 61) mice via intra-peritoneal injection reduces the accumulation of α-syn, where a single injection of ApoB¹¹:2-O′OMe siα-syn (50 μg) was delivered to 4-month-old α-syn tg or non-tg mice and 1 month later mice were sacrificed and analyzed for accumulation of α-syn (Syn211) (FIG. A-C) and P-syn (pSer129) (FIG. D-F). Quantification of immunohistochemistry was performed with ImageJ™ by counting positive cells. *-indicates p<0.05 compared to si control treated α-syn tg mice. ***-indicates p<0.001 compared to si-control treated α-syn tg mice. One-way ANOVA with poshoc Tukey-Kramer.

### REFERENCES

1. Abbott, N. J., L. Ronnback, and E. Hansson, Astrocyte-endothelial interactions at the blood-brain barrier. Nat Rev Neurosci, 2006. 7(1): p. 41-53.

2. Begley, D. J., ABC transporters and the blood-brain barrier. Curr Pharm Des, 2004. 10(12): p. 1295-312.

3. Begley, D. J. and M. W. Brightman, Structural and functional aspects of the blood-brain barrier. Prog Drug Res, 2003. 61: p. 39-78.

4. Spencer, B., et al., ESCRT-mediated uptake and degradation of brain-targeted alphα-synuclein single chain antibody attenuates neuronal degeneration in vivo. Mol Ther, 2014. 22(10): p. 1753-67.

5. Spencer, B., et al., Peripheral delivery of a CNS targeted, metalo-protease reduces abeta toxicity in a mouse model of Alzheimer's disease. PLoS One, 2011. 6(1): p. e16575.

6. Spencer, B., et al., Systemic Central Nervous System (CNS)-targeted Delivery of Neuropeptide Y (NPY) Reduces Neurodegeneration and Increases Neural Precursor Cell Proliferation in a Mouse Model of Alzheimer Disease. J Biol Chem, 2016. 291(4): p. 1905-20.

7. Spencer, B., et al., A brain-targeted, modified neurosin (kallikrein-6) reduces alphα-synuclein accumulation in a mouse model of multiple system atrophy. Mol Neurodegener, 2015. 10(1): p. 48.

8. Spencer, B., et al., A neuroprotective brain-penetrating endopeptidase fusion protein ameliorates Alzheimer disease pathology and restores neurogenesis. J Biol Chem, 2014. 289(25): p. 17917-31.

9. Spencer, B. J. and I. M. Verma, Targeted delivery of proteins across the blood-brain barrier. Proc Natl Acad Sci U S A, 2007. 104(18): p. 7594-9.

10. Yang, C. Y., et al., Sequence, structure, receptor-binding domains and internal repeats of human apolipoprotein B-100. Nature, 1986. 323(6090): p. 738-42.

11. Knott, T. J., et al., Complete cDNA and derived protein sequence of human apolipoprotein B-100. Nucleic Acids Res, 1986. 14(18): p. 7501-3.

12. Javed, H., et al., Development of Nonviral Vectors Targeting the Brain as a Therapeutic Approach For Parkinson's Disease and Other Brain Disorders. Mol Ther, 2016. 24(4): p. 746-58.

13. Iwai, A., et al., The precursor protein of non-A beta component of Alzheimer's disease amyloid is a presynaptic protein of the central nervous system. Neuron, 1995. 14(2): p. 467-75.

14. Valera, E. and E. Masliah, Immunotherapy for neurodegenerative diseases: Focus on alphα-synucleinopathies. Pharmacol Ther, 2013.

15. Chandra, S., et al., Double-knockout mice for alpha- and betα-synucleins: effect on synaptic functions. Proc Natl Acad Sci U S A, 2004. 101(41): p. 14966-71.

16. Eller, M. and D. R. Williams, alpha-Synuclein in Parkinson disease and other neurodegenerative disorders. Clin Chem Lab Med, 2011. 49(3): p. 403-8.

17. Price, D.L., et al., Alterations in mGluR5 expression and signaling in Lewy body disease and in transgenic models of alphα-synucleinopathy--implications for excitotoxicity. PLoS One, 2010. 5(11): p. e14020.

18. Overk, C. R. and E. Masliah, Pathogenesis of synaptic degeneration in Alzheimer's disease and Lewy body disease. Biochem Pharmacol, 2014. 88(4): p. 508-16.

19. Spencer, B., et al., alpha-Synuclein interferes with the ESCRT-III complex contributing to the pathogenesis of Lewy body disease. Hum Mol Genet, 2016. 25(6): p. 1100-15.

20. Lim, H. Y., et al., Lymphatic vessels are essential for the removal of cholesterol from peripheral tissues by SR-BI-mediated transport of HDL. Cell Metab, 2013. 17(5): p. 671-84.

21. Wolfrum, C., et al., Mechanisms and optimization of in vivo delivery of lipophilic siRNAs. Nat Biotechnol, 2007. 25(10): p. 1149-57.

22. Hammond, S. M., Dicing and slicing: the core machinery of the RNA interference pathway. FEBS Lett, 2005. 579(26): p. 5822-9.

23. Layzer, J. M., et al., In vivo activity of nuclease-resistant siRNAs. RNA, 2004. 10(5): p. 766-71.

24. Soutschek, J., et al., Therapeutic silencing of an endogenous gene by systemic administration of modified siRNAs. Nature, 2004. 432(7014): p. 173-8.

25. Bartlett, D. W. and M. E. Davis, Insights into the kinetics of siRNA-mediated gene silencing from live-cell and live-animal bioluminescent imaging. Nucleic Acids Res, 2006. 34(1): p. 322-33.

26. Stein, C. A. and D. Castanotto, FDA-Approved Oligonucleotide Therapies in 2017. Mol Ther, 2017. 25(5): p. 1069-1075.

27. Wang, T., et al., Challenges and opportunities for siRNA-based cancer treatment. Cancer Lett, 2017. 387: p. 77-83.

28. Lentz, T. L., Structure-function relationships of curaremimetic neurotoxin loop 2 and of a structurally similar segment of rabies virus glycoprotein in their interaction with the nicotinic acetylcholine receptor. Biochemistry, 1991. 30(45): p. 10949-57.

29. Tian, X., et al., LRP-1-mediated intracellular antibody delivery to the Central Nervous System. Sci Rep, 2015. 5: p. 11990.

30. Bruggmann, D., et al., Multiple nicotinic acetylcholine receptor alpha-subunits are expressed in the arterial system of the rat. Histochem Cell Biol, 2002. 118(6): p. 441-7.

31. Bruggmann, D., et al., Rat arteries contain multiple nicotinic acetylcholine receptor alpha-subunits. Life Sci, 2003. 72(18-19): p. 2095-9.

32. Suzuki, T., et al., Microglial alpha7 nicotinic acetylcholine receptors drive a phospholipase C/IP3 pathway and modulate the cell activation toward a neuroprotective role. J Neurosci Res, 2006. 83(8): p. 1461-70.

33. Isaias, I. U., et al., Nicotinic acetylcholine receptor density in cognitively intact subjects at an early stage of Parkinson's disease. Front Aging Neurosci, 2014. 6: p. 213.

34. Wunner, W. H., K. J. Reagan, and H. Koprowski, Characterization of saturable binding sites for rabies virus. J Virol, 1984. 50(3): p. 691-7.

35. Duffy, K.R., W. M. Pardridge, and R. G. Rosenfeld, Human blood-brain barrier insulin-like growth factor receptor. Metabolism, 1988. 37(2): p. 136-40.

36. Friden, P. M., et al., Anti-transferrin receptor antibody and antibody-drug conjugates cross the blood-brain barrier. Proc Natl Acad Sci U S A, 1991. 88(11): p. 4771-5.

37. Pardridge, W. M., et al., Human insulin receptor monoclonal antibody undergoes high affinity binding to human brain capillaries in vitro and rapid transcytosis through the blood-brain barrier in vivo in the primate. Pharm Res, 1995. 12(6): p. 807-16.

38. Coloma, M. J., et al., Transport across the primate blood-brain barrier of a genetically engineered chimeric monoclonal antibody to the human insulin receptor. Pharm Res, 2000. 17(3): p. 266-74.

39. Bickel, U., T. Yoshikawa, and W. M. Pardridge, Delivery of peptides and proteins through the blood-brain barrier. Adv Drug Deliv Rev, 2001. 46(1-3): p. 247-79.

40. Klebe, R. J. and F. H. Ruddle, Neuroblastoma: cell culture analysis of a differentiating stem cell system. J. Cell. Biol., 1969. 43: p. 69a.

41. Spencer, B., et al., Reducing Endogenous alpha-Synuclein Mitigates the Degeneration of Selective Neuronal Populations in an Alzheimer's Disease Transgenic Mouse Model. J Neurosci, 2016. 36(30): p. 7971-84.

42. Masliah, E., et al., Dopaminergic loss and inclusion body formation in alpha-synuclein mice: implications for neurodegenerative disorders. Science, 2000. 287(5456): p. 1265-9.

43. Rockenstein, E., et al., Differential neuropathological alterations in transgenic mice expressing alphα-synuclein from the platelet-derived growth factor and Thy-1 promoters. J Neurosci Res, 2002. 68(5): p. 568-78.

44. Spencer, B., et al., Beclin 1 gene transfer activates autophagy and ameliorates the neurodegenerative pathology in alphα-synuclein models of Parkinson's and Lewy body diseases. J Neurosci, 2009. 29(43): p. 13578-88.

45. Crews, L., et al., Selective molecular alterations in the autophagy pathway in patients with Lewy body disease and in models of alphα-synucleinopathy. PLoS One, 2010. 5(2): p. e9313.

46. Spencer, B., et al., Lentivirus mediated delivery of neurosin promotes clearance of wild-type alphα-synuclein and reduces the pathology in an alpha-synuclein model of LBD. Mol Ther, 2013. 21(1): p. 31-41.

47. Spencer, B., et al., Systemic peptide mediated delivery of an siRNA targeting alphα-syn in the CNS ameliorates the neurodegenerative process in a transgenic model of Lewy body disease. Neurobiol Dis, 2019. 127: p. 163-177.

A number of embodiments of the invention have been described. Nevertheless, it can be understood that various modifications may be made without departing from the spirit and scope of the invention. Accordingly, other embodiments are within the scope of the following claims.

What is claimed is:

1. A chimeric or synthetic peptide, comprising:
(a) a first peptide comprising an amino acid sequence as set forth in SEQ ID NO:2, or a mimetic or peptidomimetic thereof;
(b) a second peptide comprising at least 2, 3, 4, 5, 6, 7, 8, 9 or 10 or more or a plurality of amino acids or mimetics or peptidomimetics thereof capable of functioning as a flexible linker,
and optionally the second peptide comprises at least 2, 3, 4, 5, 6, 7, 8, 9 or 10 or more or a plurality of glycines, or mixture of glycines, serines, valines, alanines, isoleucines, leucines or mimetics or peptidomimetics thereof,
and optionally the second peptide comprises or consists of five glycines or mixture of glycines and serines, or mixture of glycines, serines, valines, alanines, isoleucines, leucines or mimetics or peptidomimetics thereof,
and optionally the second peptide comprises or consists of GSGGG (SEQ ID NO:7), GGSGG (SEQ ID NO:8), GGGGS (SEQ ID NO:9), GGSGGG (SEQ ID NO:10), GSGGGG (SEQ ID NO:11), GGSGGGS (SEQ ID NO:12), GGSGSGS (SEQ ID NO:13), GGSGSGSGG (SEQ ID NO:14), GGSGGGSGG (SEQ ID NO:15), GGSGGGSGGGS (SEQ ID NO:16) or GGSGGGSGSGS (SEQ ID NO:17) or mimetics or peptidomimetics thereof; and
(c) a positively charged third peptide comprising at least 2, 3, 4, 5, 6, 7, 8, 9, 10, 11, 12, 13 or 14 or more positively charged amino acids, or mimetics or peptidomimetics thereof, wherein optionally the positively charged amino acids comprise an arginine or a mimetic or peptidomimetic thereof, and optionally the third peptide comprises nine arginines or mimetics or peptidomimetics thereof,
wherein optionally the chimeric or synthetic peptide comprises or consists of: RLTRKRGLKLAGGGGGRRRRRRRRR (SEQ ID NO:3) or a mimetic or peptidomimetic thereof.

2. The chimeric or synthetic peptide of claim 1, wherein the chimeric or synthetic peptide further comprises a nucleic acid, or a negatively charged payload,
wherein optionally the nucleic acid or the negatively charged payload is electrostatically bound, or bound by a charge, to the positively charged third peptide, or the nucleic acid or negatively charged payload is bound by a chemical bond to the chimeric or synthetic peptide.

3. The chimeric or synthetic peptide of claim 1, wherein the first peptide is linked to the second peptide by a peptide bond; or, the second peptide and the positively charged third peptide are linked together by a peptide bond; or, the first peptide, the second peptide and the positively charged third peptide are all linked together by a peptide bond.

4. The chimeric or synthetic peptide of claim 1, wherein the nucleic acid is or comprises an natural or a synthetic oligonucleotide,
and optionally the natural or synthetic oligonucleotide comprises an RNA or a DNA,
and optionally the natural or synthetic oligonucleotide comprises a small inhibitory RNA (siRNA), a cDNA, an mRNA, an shRNA, an miRNA or a gRNA,
and optionally the DNA comprises an antisense oligonucleotide, and optionally the antisense oligonucleotide comprises a modified or synthetic anti-sense oligonucleotide (ASO), and optionally the modified or synthetic anti-sense oligonucleotide comprises: a 2′-O-(2-MethoOxyEthyl)-oligoribonucloeotide (2′-MOE); a 2′-O-Methyl-RNA (2′-OMe) oligoribonucloeotide; a phosphorothioate DNA or an oligonucleotide phosphorothioate (OPS); a phosphorodiamidate morpholino oligomer (PMO); a tricyclo-DNA or tcDNA; a locked nucleic acid (LNA) or inaccessible RNA (or RNA nucleotide having a ribose moiety modified with an extra bridge connecting a 2′ oxygen and a 4′ carbon); or, a morpholino, 5′ methylcytosine base.

5. The chimeric or synthetic peptide of claim 1, wherein the chimeric or synthetic peptide comprises at least one synthetic amino acid, or at least two amino acid are joined by at least one non-peptide (non-amide) bond, and optionally the at least one non-peptide comprises an isopeptide bond.

6. The chimeric or synthetic peptide of claim 1, formulated as a sterile solution, or as an injectable formulation.

7. A product of manufacture comprising a chimeric or synthetic peptide of claim 1, wherein the product of manufacture comprises an implant, an ampoule or a syringe.

8. A method for treating, preventing, ameliorating and/or diagnosing a disease, infection or condition of the central nervous system (CNS) in an individual in need thereof, or causing a neuro-modulatory treatment or effect on the individual in need thereof, comprising: administering to the individual in need thereof a chimeric or synthetic peptide of claim 1, wherein the chimeric or synthetic peptide further comprises or is bound to a nucleic acid capable of treating, preventing, ameliorating and/or diagnosing the disease or condition of the CNS in the individual in need thereof.

9. The method of claim 7, wherein the disease, infection or condition of the CNS is or causes a neurodegenerative disease or a cancer.

10. The method of claim 7, wherein the neurodegenerative disease or CNS condition is: Alzheimer's disease, Lewy Body (DLB), multiple system atrophy (MSA), Gaucher's disease, amyotrophic lateral sclerosis (ALS), neuronal ceroid lipofuscinosis (NCL) (Batten disease), Parkinson's disease (PD), spinocerebellar ataxia (SCA) frontotemporal lobar degeneration (Pick's disease), a prion disease (Creutzfeldt-Jakob Disease) or Huntington's disease.

11. The method of claim 7, wherein the chimeric or synthetic peptide is formulated as a sterile injectable formulation, and optionally the chimeric or synthetic peptide is administered to the individual in need thereof by intraperitoneal (IP), intravenous (IV), intramuscular (IM) or intrathecal injection, or by inhalation.

12. A method for gene targeted editing, or genomic engineering, epigenomic engineering, genome targeting, and/or genome editing, of the CNS, comprising: administering to an individual in need thereof a chimeric or synthetic peptide of claim 1, wherein the chimeric or synthetic peptide further comprises or is bound to a nucleic acid, optionally capable of gene targeted editing or genomic engineering, epigenomic engineering, genome targeting, and/or genome editing, and optionally the gene targeted editing comprises a CRISPR nucleic acid.

13. The method of claim 11, wherein the chimeric or synthetic peptide is formulated as a sterile injectable formulation.

14. The method of claim 11, wherein the chimeric or synthetic peptide is administered to the individual in need thereof by intraperitoneal (IP), intravenous (IV), intramuscular (IM) or intrathecal injection, or by inhalation.

15. The method of claim 11, wherein the individual in need thereof has a neurodegenerative disease or a CNS condition.

16. The method of claim 15, wherein the neurodegenerative disease or the CNS condition is: Alzheimer's disease, Lewy Body (DLB), multiple system atrophy (MSA), Gaucher's disease, amyotrophic lateral sclerosis (ALS), neuronal ceroid lipofuscinosis (NCL) (Batten disease), Parkinson's disease (PD), spinocerebellar ataxia (SCA) frontotemporal lobar degeneration (Pick's disease), a prion disease (Creutzfeldt-Jakob Disease) or Huntington's disease.


SEQUENCE LISTING (17 sequences)

SEQ ID NO: 1 - protein, 42 aa, artificial sequence
  synthetic peptide
VDSSVIDALQYKLEGTTRLTRKRGLKLAGGGGGRRRRRRRRR

SEQ ID NO: 2 - protein, 11 aa, artificial sequence
  synthetic peptide
RLTRKRGLKLA

SEQ ID NO: 3 - protein, 25 aa, artificial sequence
  synthetic peptide
RLTRKRGLKLAGGGGGRRRRRRRRR

SEQ ID NO: 4 - protein, 25 aa, artificial sequence
  synthetic peptide
RLTRKRGLKLAGGGGGRRRRRRRRR

SEQ ID NO: 5 - RNA, 19 nt, artificial sequence
  synthetic polynucleotide
gacuuucaaaggccaagga

SEQ ID NO: 6 - RNA, 19 nt, artificial sequence
  synthetic polynucleotide
gggcauacugagcuaacaa

SEQ ID NO: 7 - protein, 5 aa, artificial sequence
  synthetic peptide
GSGGG

SEQ ID NO: 8 - protein, 5 aa, artificial sequence
  synthetic peptide
GGSGG

SEQ ID NO: 9 - protein, 5 aa, artificial sequence
  synthetic peptide
GGGGS

SEQ ID NO: 10 - protein, 6 aa, artificial sequence
  synthetic peptide
GGSGGG

SEQ ID NO: 11 - protein, 6 aa, artificial sequence
  synthetic peptide
GSGGGG

SEQ ID NO: 12 - protein, 7 aa, artificial sequence
  synthetic peptide
GGSGGGS

SEQ ID NO: 13 - protein, 7 aa, artificial sequence
  synthetic peptide
GGSGSGS

SEQ ID NO: 14 - protein, 9 aa, artificial sequence
  synthetic peptide
GGSGSGSGG

SEQ ID NO: 15 - protein, 9 aa, artificial sequence
  synthetic peptide
GGSGGGSGG

SEQ ID NO: 16 - protein, 11 aa, artificial sequence
  syntheitic peptide
GGSGGGSGGGS

SEQ ID NO: 17 - protein, 11 aa, artificial sequence
  synthetic peptide
GGSGGGSGSGS